(19) United States
(12) Patent Application Publication
(10) Pub. No.: US 2021/0343402 A1
(43) Pub. Date: Nov. 4, 2021

(54) HYPOGLYCEMIC EVENT PREDICTION USING MACHINE LEARNING

(71) Applicant: DexCom, Inc., San Diego, CA (US)

(72) Inventors: Giada Acciaroli, Edinburgh (GB); Mark Derdzinski, La Jolla, CA (US); Lauren Hruby Jepson, San Diego, CA (US); Andrew S. Parker, San Diego, CA (US)

(21) Appl. No.: 17/114,106

(22) Filed: Dec. 7, 2020

Related U.S. Application Data
(60) Provisional application No. 63/017,611, filed on Apr. 29, 2020.

(51) Int. Cl. G16H 40/63; G06N 3/08

(52) U.S. Cl. CPC G16H 40/63; G06N 3/08

(57) ABSTRACT

Hypoglycemic event prediction using machine learning is described. A CGM platform includes a machine learning model trained using historical time series glucose measurements of a user population. Once trained, the machine learning model predicts hypoglycemic events for users. When predicting hypoglycemic events, a time series of glucose measurements for a day time interval is received. The glucose measurements of this time series for the day time interval are provided by a CGM system worn by the user. The machine learning model predicts whether a hypoglycemic event will occur during a night time interval that is subsequent to the day time interval by processing the time series of glucose measurements using the trained machine learning model. The hypoglycemic event prediction is then output, such as via communication and/or display of a notification about the hypoglycemic event prediction.

### INCORPORATION BY REFERENCE TO RELATED APPLICATION

Any and all priority claims identified in the Application Data Sheet, or any correction thereto, are hereby incorporated by reference under 37 CFR 1.57. This application claims the benefit of U.S. Provisional Patent Application No. 63/017,611, filed Apr. 29, 2020, and titled “Hypoglycemic Event Prediction Using Machine Learning”. The aforementioned application is incorporated by reference herein in its entirety, and is hereby expressly made a part of this specification.

### BACKGROUND

Diabetes is a metabolic condition affecting hundreds of millions of people, and is one of the leading causes of death worldwide. For people living with diabetes, access to treatment is critical to their survival. With proper treatment, serious damage to the heart, blood vessels, eyes, kidneys, and nerves, due to diabetes can be largely avoided. Proper treatment for a person with Type I diabetes generally involves monitoring glucose levels throughout the day and regulating those levels—with some combination of insulin, eating, and exercise—so that the levels stay within a desired range. With advances in medical technologies a variety of systems for monitoring glucose levels have been developed. While monitoring a person's current glucose level is useful for deciding how to treat diabetes, knowing what the person's glucose levels will be in the future is more useful. This is because it allows the person or a caregiver to take actions to mitigate potentially adverse health conditions, tied to changing glucose levels (e.g., hyperglycemia or hypoglycemia), before such health conditions occur.

Hypoglycemia is a condition in which a person's glucose level is low, as compared to hyperglycemia which occurs when a person's glucose level is high. A glucose level usually is considered “low” when it falls below 70 mg/dl, although low may be defined by different thresholds. Hypoglycemia is a concern due to its potentially negative side effects, which can include confusion, abnormal behavior (e.g., the inability to complete routine tasks), visual disturbances (e.g., blurred vision), seizures, and loss of consciousness. In severe cases, hypoglycemia can even lead to death. It is estimated that nearly half of all episodes of hypoglycemia, and more than half of all severe episodes, occur at night, during sleep. Hypoglycemia that occurs at night may be referred to as “nocturnal” or “nighttime” hypoglycemia. Conventional systems are unable to accurately predict whether a person will experience an episode of hypoglycemia during a given night and thus also to advise the person how he or she should behave or actions to take to mitigate nighttime hypoglycemia.

### SUMMARY

To overcome these problems, hypoglycemic event prediction using machine learning is leveraged. Given the number of people that wear continuous glucose monitoring (CGM) systems and because CGM systems produce measurements continuously, a CGM platform that provides a CGM system with a sensor for detecting glucose levels, and maintains measurements produced by such a system may have an enormous amount of data, e.g., tens of millions of patient days' worth of measurements. However, this amount of data is practically, if not actually, impossible for a human to process to reliably identify patterns of a robust number of state spaces.

In one or more implementations, a CGM platform includes a machine learning model trained using historical time series glucose measurements of a user population, where the glucose measurements are provided by CGM systems worn by users of the user population. Although in some implementations the machine learning model may be limited to receiving time series glucose measurements as input, in one or more implementations, the machine learning model also receives, as input, additional data describing one or more other aspects that impact a person's glucose in the future, such as application usage activity, insulin administered, exercise, and so forth. Once trained, the machine learning model predicts hypoglycemic events for users. When predicting hypoglycemic events, a time series of glucose measurements for a day time interval is received. The glucose measurements of this time series for the day time interval are provided by a CGM system worn by the user. The machine learning model predicts whether a hypoglycemic event will occur during a night time interval that is subsequent to the day time interval by processing the time series of glucose measurements using the trained machine learning model. The hypoglycemic event prediction is then output, such as via communication and/or display of a notification about the hypoglycemic event prediction. For example, the hypoglycemic event prediction corresponds to a positive result if the hypoglycemic event is predicted to occur during the night time interval by the machine learning model or a negative result if the hypoglycemic event is predicted not to occur during the night time interval by the machine learning model.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ techniques described herein.

FIG. 2 depicts an example of the continuous glucose monitoring (CGM) system of FIG. 1 in greater detail.

FIG. 3 depicts an example implementation in which CGM device data, including glucose measurements, is routed to different systems in connection with hypoglycemic event prediction.

FIG. 4 depicts an example implementation of the hypoglycemic event prediction system of FIG. 3 in greater detail to predict whether a hypoglycemic event will occur during an upcoming night time interval using machine learning.

FIG. 5 depicts an example implementation in which the described machine learning model generates a hypoglycemic event prediction in accordance with one or more implementations.

FIG. 6 depicts an additional example implementation in which the described machine learning model generates a hypoglycemic event prediction in accordance with one or more implementations.

FIG. 7 depicts an additional example implementation of the hypoglycemic event prediction system of FIG. 3 in greater detail to output notifications based on a hypoglycemic event prediction.

FIG. 8 depicts example implementations of user interfaces displayed for notifying a user based on predictions of hypoglycemic events occurring during a night time interval.

FIG. 9 depicts an example implementation of the hypoglycemic event prediction system in greater detail in which a machine learning model is trained to predict whether a hypoglycemic event will occur during a night time interval.

FIG. 10 illustrates an example implementation of training data generated by the model manager for training the described machine learning model.

FIG. 11 depicts a procedure in an example implementation in which a machine learning model predicts whether a hypoglycemic event will occur during a night time interval.

FIG. 12 depicts a procedure in an example implementation in which a machine learning model is trained to predict hypoglycemic events based on historical time series glucose measurements of a user population.

FIG. 13 illustrates an example system that includes an example computing device that is representative of one or more computing systems and/or devices that may implement the various techniques described herein.

### DETAILED DESCRIPTION

### Overview

Hypoglycemic event prediction using machine learning is described. In one or more implementations, a continuous glucose monitoring (CGM) platform includes a machine learning model trained, using historical time series glucose measurements of a user population, to predict whether a hypoglycemic event will occur during a night time interval. The glucose measurements of the user population and the individual user may be provided by CGM systems worn by users of the user population and the individual user. By obtaining measurements produced by these CGM systems and maintaining the measurements, the CGM platform may have an enormous amount of data, e.g., tens of millions of patient days' worth of measurements. Conventional machine learning models may not be able to model some of the patterns observed in this wealth of historical data in order to accurately predict hypoglycemic events during night time intervals. Moreover, the time series glucose measurements described herein correspond to a time-ordered sequence of the glucose measurements, which may otherwise be referred to as a “glucose trace”. It is to be appreciated, therefore, that such, time series glucose measurements used to both build the machine learning model, and subsequently used as input to predict night time hypoglycemia, correspond to sequential streams of glucose measurements which can be distinguished from conventional systems which utilize glucose “features as inputs” to prediction models.

Once the machine learning model is trained, it is used to predict whether an episode of hypoglycemia will occur for a person during a night time interval, e.g., while the person is sleeping over the next number of hours. When predicting whether a hypoglycemic event will occur during the night for a particular user, a time series of glucose measurements up to a time of the prediction is received from the CGM system worn by the user. For example, a time series of glucose measurements for a day time interval of a current day are utilized by the machine learning model to predict whether a hypoglycemic event will occur during a night time interval that is subsequent to the day time interval. The machine learning model generates this prediction based on its training with the historical time series glucose measurements of the user population.

Notably, many factors may affect the glucose levels of users during the night, including exercise, food consumption, and insulin (e.g., administered via an insulin pen). For example, exercise may increase insulin sensitivity, and thus a user that is receiving insulin to treat Diabetes is likely to drop her glucose level much more if she exercises during the course of a day because this will cause her to be more “sensitive” and less “resistant” to the insulin dose she normally receives. Thus, an increase in the amount of exercise performed by the user, without a reduction in insulin administered, may result in night time hypoglycemia. As another example, eating food, and particularly carbohydrates, will cause an increase in the glucose levels of the user. As another example, in some cases a user may become aware of her glucose level, and then take various actions to mitigate night time hypoglycemia, such as by consuming a piece of fruit before bedtime. However, many of these actions and behaviors by users are hidden to conventional prediction systems, and are thus unaccounted for when generating hypoglycemic event predictions.

To solve this problem, in one or more implementations the machine learning model also receives, as input, additional data describing one or more other aspects that impact a person's glucose in the future. The additional data may be correlated in time to the time series of glucose measurements, e.g., based on timestamps associated with the additional data. Such additional data may include, by way of example and not limitation, application usage data (e.g., clickstream data describing user interfaces displayed and user interactions with applications via the user interfaces), accelerometer data of a mobile device or smart watch (e.g., indicating that that the person has viewed a user interface of the device and thus has likely seen an alert or information related to her glucose levels), data describing insulin administered (e.g., timing and insulin doses), food consumed (e.g., timing of food consumption, type of food, and/or amount of carbohydrates consumed), activity data from various sensors (e.g., step data, workouts performed, or other data indicative of user activity or exercise), stress, and so forth. The machine learning model, in this case, is also trained using historical additional data of the user population. Thus, the accuracy of the predictions generated by the machine learning model are increased by utilizing both the time series glucose measurements and the additional data to generate the predictions. For example, the machine learning model can be trained to learn patterns associated with application usage activity, exercise, food consumption, and insulin doses administered, and thus adjust the hypoglycemic event predictions accordingly.

In one or more implementations, the additional data received as input by the machine learning model is associated with an application of the CGM platform. For example, an application of the CGM platform may be executed at a user's computing device (e.g., a smartphone or smartwatch) to display the glucose measurements to the user, e.g., in a user interface of an application of the CGM platform. The additional data, in this instance, may correspond to screen views or user selections of different controls of the CGM application. Such application usage data enables the machine learning model to learn whether the user is aware of her current glucose condition, which may indicate that the user has taken a mitigating action to correct the glucose condition. For example, if the user looks at the CGM application shortly before going to bed and notices that her blood glucose levels are dropping, she may take a mitigating action to prevent night time hypoglycemia, such as by eating a piece of fruit. This mitigating action may affect the accuracy of the hypoglycemic event prediction. For example, if the system had predicted the occurrence of night time hypoglycemia, then the mitigating action may prevent the occurrence of night time hypoglycemia causing the prediction to be inaccurate. As such, the machine learning model can learn patterns associated with mitigating actions performed by the user, and then adjust the hypoglycemic event predictions accordingly.

In one or more implementations, the accuracy of the hypoglycemic event prediction may be further increased by obtaining glucose measurements for the user during a period of inactivity. In this case, the system may output instructions for the user to follow in order to more accurately predict whether hypoglycemia will occur during the night time interval. By way of example, and not limitation, the instructions may instruct, for a time period (e.g., 30 minutes), the user not to eat, the user to lessen his or her activity (e.g., no exercise, no vigorous activity, keep heart rate below a certain level), the user to continue wearing particular devices to monitor various physiological signals, and so forth. During this time period of relative inactivity, the machine learning model may obtain the time series glucose measurements for the period of inactivity in order to predict the occurrence of hypoglycemia during the night time interval.

The hypoglycemic event prediction is then output, such as for generating a notification about whether an episode of hypoglycemia will occur for the person during the night time interval. This notification may be communicated over a network to one or more computing devices, such as a computing device associated with the user (e.g., for output via an application of the CGM platform) or a computing device associated with a guardian of the user (e.g., the user's parent). For example, if the machine learning model predicts that the user is likely to experience nighttime hypoglycemia during the upcoming night time interval, then a notification is output indicating this is the case. In one or more implementations, the described system can additionally output one or more recommendations for mitigating the predicted hypoglycemia, such as to drink a glass of juice before sleep, eat a piece of fruit before sleep, set an alarm for a certain time to wake up and drink juice or eat fruit, and so forth. On the other hand, if the machine learning model predicts that the user is not likely to experience hypoglycemia during the night time interval then the described system can output a notification indicating that this is the case and/or that no mitigating actions need to be taken. In this case, the user can then go to sleep with confidence that a hypoglycemic event will not occur that night.

In one or more implementations, the CGM platform may adjust various settings of the CGM system during the night time interval based on hypoglycemic event prediction, such as by adjusting the glucose alert settings for the night time interval if a negative result is predicted. For example, a threshold for a low glucose alert may be adjusted by raising the threshold during the night time interval when the machine learning model predicts that the user will not experience an episode of hypoglycemia. This has the effect of triggering a low glucose alert earlier than usual in order to give the user more time to mitigate their low glucose level in the event that a hypoglycemic event is experienced after the system predicts that a hypoglycemic event will not occur. The adjusted settings may also override customized alert settings which may have been modified by the user. Notably, adjusting the system settings may safeguard against the possibility that the user experiences an episode of hyperglycemia during the night in the event that the prediction is incorrect due to unseen factors.

By accurately predicting whether a hypoglycemic event will occur during a night time interval and notifying users, the described machine learning model allows actions to be taken by users to mitigate the occurrence of hypoglycemia at night before it occurs. Advantageously, the more accurate and timely predictions of night time hypoglycemia provided by the described machine learning model allow users and various other parties to make better informed decisions regarding how to prevent the harmful effects of night time hypoglycemia.

In the following discussion, an example environment is first described that may employ the techniques described herein. Example implementation details and procedures are then described which may be performed in the example environment as well as other environments. Performance of the example procedures is not limited to the example environment and the example environment is not limited to performance of the example procedures.

### Example Environment

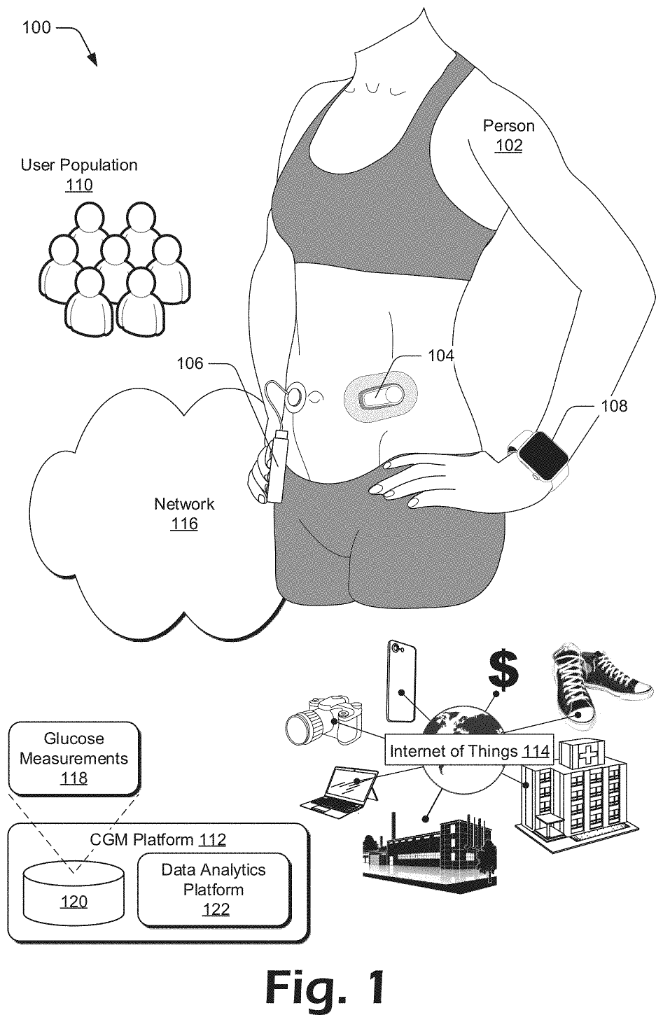

FIG. 1 is an illustration of an environment 100 in an example implementation that is operable to employ hypoglycemic event prediction using machine learning described herein. The illustrated environment 100 includes person 102, who is depicted wearing a continuous glucose monitoring (CGM) system 104, insulin delivery system 106, and computing device 108. The illustrated environment 100 also includes other users in a user population 110 of the CGM system, CGM platform 112, and Internet of Things 114 (IoT 114). The CGM system 104, insulin delivery system 106, computing device 108, user population 110, CGM platform 112, and IoT 114 are communicatively coupled, including via a network 116.

Alternately or additionally, one or more of the CGM system 104, the insulin delivery system 106, and the computing device 108 may be communicatively coupled in other ways, such as using one or more wireless communication protocols or techniques. By way of example, the CGM system 104, the insulin delivery system 106, and the computing device 108 may communicate with one another using one or more of Bluetooth (e.g., Bluetooth Low Energy links), near-field communication (NFC), 5G, and so forth. The CGM system 104, the insulin delivery system 106, and the computing device 108 may leverage these types of communication to form a closed-loop system between one another. In this way, the insulin delivery system 106 may deliver insulin based on sequences of glucose measurements in real-time as glucose measurements are obtained by the CGM system 104 and as future glucose measurements are predicted.

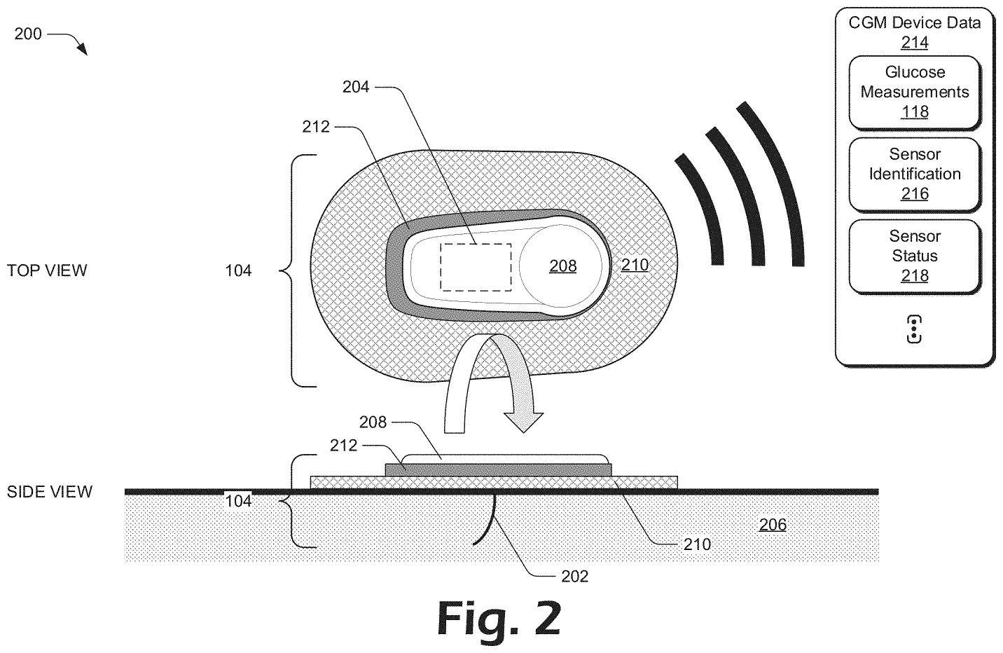

In accordance with the described techniques, the CGM system 104 is configured to monitor glucose of the person 102 continuously. The CGM system 104 may be configured with a CGM sensor, for instance, that continuously detects analytes indicative of the person 102's glucose and enables generation of glucose measurements. In the illustrated environment 100 these measurements are represented as glucose measurements 118. This functionality along with further aspects of the CGM system 104's configuration are discussed in more detail in relation to FIG. 2.

In one or more implementations, the CGM system 104 transmits the glucose measurements 118 to the computing device 108, such as via a wireless connection. The CGM system 104 may communicate these measurements in real-time, e.g., as they are produced using a CGM sensor. Alternately or in addition, the CGM system 104 may communicate the glucose measurements 118 to the computing device 108 at set time intervals, e.g., every 30 seconds, every minute, every 5 minutes, every hour, every 6 hours, every day, and so forth. Further still, the CGM system 104 may communicate these measurements responsive to a request from the computing device 108, e.g., communicated to the CGM system 104 when the computing device 108 predicts the person 102's upcoming glucose level, causes display of a user interface having information about the person 102's glucose level, updates such a display, and so forth. Accordingly, the computing device 108 may maintain the glucose measurements 118 of the person 102 at least temporarily, e.g., in computer-readable storage media of the computing device 108.

Although illustrated as a wearable device (e.g., a smart watch), the computing device 108 may be configured in a variety of ways without departing from the spirit or scope of the described techniques. By way of example and not limitation, the computing device 108 may be configured as a different type of mobile device (e.g., a mobile phone or tablet device). In one or more implementations, the computing device 108 may be configured as a dedicated device associated with the CGM platform 112, e.g., with functionality to obtain the glucose measurements 118 from the CGM system 104, perform various computations in relation to the glucose measurements 118, display information related to the glucose measurements 118 and the CGM platform 112, communicate the glucose measurements 118 to the CGM platform 112, and so forth. In contrast to implementations where the computing device 108 is configured as a mobile phone, however, the computing device 108 may not include some functionality available with mobile phone or wearable configurations when configured as a dedicated CGM device, such as the ability to make phone calls, camera functionality, the ability to utilize social networking applications, and so on.

Additionally, the computing device 108 may be representative of more than one device in accordance with the described techniques. In one or more scenarios, for instance, the computing device 108 may correspond to both a wearable device (e.g., a smart watch) and a mobile phone. In such scenarios, both of these devices may be capable of performing at least some of the same operations, such as to receive the glucose measurements 118 from the CGM system 104, communicate them via the network 116 to the CGM platform 112, display information related to the glucose measurements 118, and so forth. Alternately or in addition, different devices may have different capabilities that other devices do not have or that are limited through computing instructions to specified devices.

In the scenario where the computing device 108 corresponds to a separate smart watch and a mobile phone, for instance, the smart watch may be configured with various sensors and functionality to measure a variety of physiological markers (e.g., heartrate, breathing, rate of blood flow, and so on) and activities (e.g., steps) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images of meals used to predict future glucose levels and an amount of computing resources (e.g., battery and processing speed) that enables the mobile phone to more efficiently carry out computations in relation to the glucose measurements 118. Even in scenarios where a smart watch is capable of carrying out such computations, computing instructions may limit performance of those computations to the mobile phone so as not to burden both devices and to utilize available resources efficiently. To this extent, the computing device 108 may be configured in different ways and represent different numbers of devices than discussed herein without departing from the spirit and scope of the described techniques.

As mentioned above, the computing device 108 communicates the glucose measurements 118 to the CGM platform 112. In the illustrated environment 100, the glucose measurements 118 are shown stored in storage device 120 of the CGM platform 112. The storage device 120 may represent one or more databases and also other types of storage capable of storing the glucose measurements 118. The storage device 120 also stores a variety of other data. In accordance with the described techniques, for instance, the person 102 corresponds to a user of at least the CGM platform 112 and may also be a user of one or more other, third party service providers. To this end, the person 102 may be associated with a username and be required, at some time, to provide authentication information (e.g., password, biometric data, a telemedicine service, and so forth) to access the CGM platform 112 using the username This information, along with other information about the user, may be maintained in the storage device 120, including, for example, demographic information describing the person 102, information about a health care provider, payment information, prescription information, determined health indicators, user preferences, account information for other service provider systems (e.g., a service provider associated with a wearable, social networking systems, and so on), and so forth.

The storage device 120 also maintains data of the other users in the user population 110. Given this, the glucose measurements 118 in the storage device 120 include the glucose measurements from a CGM sensor of the CGM system 104 worn by the person 102 and also include glucose measurements from CGM sensors of CGM systems worn by persons corresponding to the other users in the user population 110. It follows also that the glucose measurements 118 of these other users are communicated by their respective devices via the network 116 to the CGM platform 112 and that these other users have respective user profiles with the CGM platform 112.

The data analytics platform 122 represents functionality to process the glucose measurements 118—alone and/or along with other data maintained in the storage device 120—to generate a variety of predictions, such as by using various machine learning models. Based on these predictions, the CGM platform 112 may provide notifications in relation to the predictions, such as alerts, recommendations, or other information based on the predictions. For instance, the CGM platform 112 may provide the notifications to the user, to a medical professional associated with the user, and so forth. Although depicted as separate from the computing device 108, portions or an entirety of the data analytics platform 122 may alternately or additionally be implemented at the computing device 108. The data analytics platform 122 may also generate these predictions using additional data obtained via the IoT 114.

In one or more implementations, the data analytics platform 122 is configured to process glucose measurements 118 obtained over a first time interval in order to predict whether or not a user will have a hypoglycemic event during an upcoming time interval in the future. For example, the data analytic platform can process glucose measurements 118 obtained during the course of a day, in order to predict whether or not the user will have a hypoglycemic event during the night. The prediction can then be output to the user, e.g., via computing device 108, so that the user can take an appropriate action. If the prediction indicates that the user will not have a hypoglycemic event during the night, for instance, the user can then go to sleep with confidence that a hypoglycemic event will not occur that night. In contrast, if the prediction indicates that the user will experience a hypoglycemic event during the night, then the user may take a mitigating action to reduce the probability of the hypoglycemic event occurring, such as by drinking a glass of juice before sleep, eating a piece of fruit before sleep, setting an alarm for a certain time to wake up and drink juice or eat fruit, and so forth. Although depicted as separate from the computing device 108, portions or an entirety of the data analytics platform 122, may alternately or additionally be implemented at the computing device 108. The data analytics platform 122 may also generate these predictions using additional data obtained via the IoT 114.

It is to be appreciated that the IoT 114 represents various sources capable of providing data that describes the person 102 and the person 102's activity as a user of one or more service providers and activity with the real world. By way of example, the IoT 114 may include various devices of the user, e.g., cameras, mobile phones, laptops, and so forth. To this end, the IoT 114 may provide information about interaction of the user with various devices, e.g., interaction with web-based applications, photos taken, communications with other users, and so forth. The IoT 114 may also include various real-world articles (e.g., shoes, clothing, sporting equipment, appliances, automobiles, etc.) configured with sensors to provide information describing behavior, such as steps taken, force of a foot striking the ground, length of stride, temperature of a user (and other physiological measurements), temperature of a user's surroundings, types of food stored in a refrigerator, types of food removed from a refrigerator, driving habits, and so forth. The IoT 114 may also include third parties to the CGM platform 112, such as medical providers (e.g., a medical provider of the person 102) and manufacturers (e.g., a manufacturer of the CGM system 104, the insulin delivery system 106, or the computing device 108) capable of providing medical and manufacturing data, respectively, that can be leveraged by the data analytics platform 122. Certainly, the IoT 114 may include devices and sensors capable of providing a wealth of data for use in connection with glucose prediction using machine learning and time series glucose measurements without departing from the spirit or scope of the described techniques. In the context of measuring glucose, e.g., continuously, and obtaining data describing such measurements, consider the following discussion of FIG. 2.

FIG. 2 depicts an example implementation 200 of the CGM system 104 of FIG. 1 in greater detail. In particular, the illustrated example 200 includes a top view and a corresponding side view of the CGM system 104.

The CGM system 104 is illustrated to include a sensor 202 and a sensor module 204. In the illustrated example 200, the sensor 202 is depicted in the side view having been inserted subcutaneously into skin 206, e.g., of the person 102. The sensor module 204 is depicted in the top view as a dashed rectangle. The CGM system 104 also includes a transmitter 208 in the illustrated example 200. Use of the dashed rectangle for the sensor module 204 indicates that it may be housed or otherwise implemented within a housing of the transmitter 208. In this example 200, the CGM system 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206 via the attachment mechanism 212. Additionally or alternately, the transmitter 208 may be incorporated as part of the application assembly, such that the sensor 202, the adhesive pad 210, the attachment mechanism 212, and the transmitter 208 (with the sensor module 204) can all be applied at once to the skin 206. In one or more implementations, this application assembly is applied to the skin 206 using a separate applicator (not shown). This application assembly may also be removed by peeling the adhesive pad 210 off of the skin 206. It is to be appreciated that the CGM system 104 and its various components as illustrated are simply one example form factor, and the CGM system 104 and its components may have different form factors without departing from the spirit or scope of the described techniques.

In operation, the sensor 202 is communicatively coupled to the sensor module 204 via at least one communication channel which can be a “wireless” connection or a “wired” connection. Communications from the sensor 202 to the sensor module 204 or from the sensor module 204 to the sensor 202 can be implemented actively or passively and these communications can be continuous (e.g., analog) or discrete (e.g., digital).

The sensor 202 may be a device, a molecule, and/or a chemical which changes or causes a change in response to an event which is at least partially independent of the sensor 202. The sensor module 204 is implemented to receive indications of changes to the sensor 202 or caused by the sensor 202. For example, the sensor 202 can include glucose oxidase which reacts with glucose and oxygen to form hydrogen peroxide that is electrochemically detectable by the sensor module 204 which may include an electrode. In this example, the sensor 202 may be configured as or include a glucose sensor configured to detect analytes in blood or interstitial fluid that are indicative of glucose level using one or more measurement techniques.

In another example, the sensor 202 (or an additional sensor of the CGM system 104—not shown) can include a first and second electrical conductor and the sensor module 204 can electrically detect changes in electric potential across the first and second electrical conductor of the sensor 202. In this example, the sensor module 204 and the sensor 202 are configured as a thermocouple such that the changes in electric potential correspond to temperature changes. In some examples the sensor module 204 and the sensor 202 are configured to detect a single analyte, e.g., glucose. In other examples, the sensor module 204 and the sensor 202 are configured to detect multiple analytes, e.g., sodium, potassium, carbon dioxide, and glucose. Alternately or additionally, the CGM system 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate the glucose measurements 118 based on the communications with the sensor 202 that are indicative of the above-discussed changes. Based on these communications from the sensor 202, the sensor module 204 is further configured to generate CGM device data 214. The CGM device data 214 is a communicable package of data that includes at least one glucose measurement 118. Alternately or additionally, the CGM device data 214 includes other data, such as multiple glucose measurements 118, sensor identification 216, sensor status 218, and so forth. In one or more implementations, the CGM device data 214 may include other information such as one or more of temperatures that correspond to the glucose measurements 118 and measurements of other analytes. It is to be appreciated that the CGM device data 214 may include a variety of data in addition to at least one glucose measurement 118 without departing from the spirit or scope of the described techniques.

In operation, the transmitter 208 may transmit the CGM device data 214 wirelessly as a stream of data to the computing device 108. Alternately or additionally, the sensor module 204 may buffer the CGM device data 214 (e.g., in memory of the sensor module 204) and cause the transmitter 208 to transmit the buffered CGM device data 214 at various intervals, e.g., time intervals (every second, every thirty seconds, every minute, every five minutes, every hour, and so on), storage intervals (when the buffered CGM device data 214 reaches a threshold amount of data or a number of instances of CGM device data 214), and so forth.

In addition to generating the CGM device data 214 and causing it to be communicated to the computing device 108, the sensor module 204 may include additional functionality in accordance with the described techniques. This additional functionality may include generating predictions of glucose levels of the person 102 in the future and communicating notifications based on the predictions, such as by communicating warnings when the predictions indicate that the person 102's level of glucose is likely to be dangerously low in the near future. This computational ability of the sensor module 204 may be advantageous especially where connectivity to services via the network 116 is limited or non-existent. In this way, a person may be alerted to a dangerous condition without having to rely on connectivity, e.g., to the Internet. This additional functionality of the sensor module 204 may also include calibrating the sensor 202 initially or on an ongoing basis as well as calibrating any other sensors of the CGM system 104.

With respect to the CGM device data 214, the sensor identification 216 represents information that uniquely identifies the sensor 202 from other sensors, such as other sensors of other CGM systems 104, other sensors implanted previously or subsequently in the skin 206, and so on. By uniquely identifying the sensor 202, the sensor identification 216 may also be used to identify other aspects about the sensor, 202 such as a manufacturing lot of the sensor 202, packaging details of the sensor 202, shipping details of the sensor 202, and so on. In this way, various issues detected for sensors manufactured, packaged, and/or shipped in a similar manner as the sensor 202 may be identified and used in different ways, e.g., to calibrate the glucose measurements 118, to notify users to change defective sensors or dispose of them, to notify manufacturing facilities of machining issues, and so forth.

The sensor status 218 represents a state of the sensor 202 at a given time, e.g., a state of the sensor at a same time one of the glucose measurements 118 is produced. To this end, the sensor status 218 may include an entry for each of the glucose measurements 118, such that there is a one-to-one relationship between the glucose measurements 118 and statuses captured in the sensor status 218 information. Generally speaking, the sensor status 218 describes an operational state of the sensor 202. In one or more implementations, the sensor module 204 may identify one of a number of predetermined operational states for a given glucose measurement 118. The identified operational state may be based on the communications from the sensor 202 and/or characteristics of those communications.

By way of example, the sensor module 204 may include (e.g., in memory or other storage) a lookup table having the predetermined number of operational states and bases for selecting one state from another. For instance, the predetermined states may include a “normal” operation state where the basis for selecting this state may be that the communications from the sensor 202 fall within thresholds indicative of normal operation, e.g., within a threshold of an expected time, within a threshold of expected signal strength, an environmental temperature is within a threshold of suitable temperatures to continue operation as expected, and so forth. The predetermined states may also include operational states that indicate one or more characteristics of the sensor 202's communications are outside of normal activity and may result in potential errors in the glucose measurements 118.

For example, bases for these non-normal operational states may include receiving the communications from the sensor 202 outside of a threshold expected time, detecting a signal strength of the sensor 202 outside a threshold of expected signal strength, detecting an environmental temperature outside of suitable temperatures to continue operation as expected, detecting that the person 102 has rolled (e.g., in bed) onto the CGM system 104, and so forth. The sensor status 218 may indicate a variety of aspects about the sensor 202 and the CGM system 104 without departing from the spirit or scope of the described techniques.

Having considered an example environment and example CGM system, consider now a discussion of some example details of the techniques for hypoglycemic event prediction using machine learning in accordance with one or more implementations.

### Hypoglycemic Event Prediction

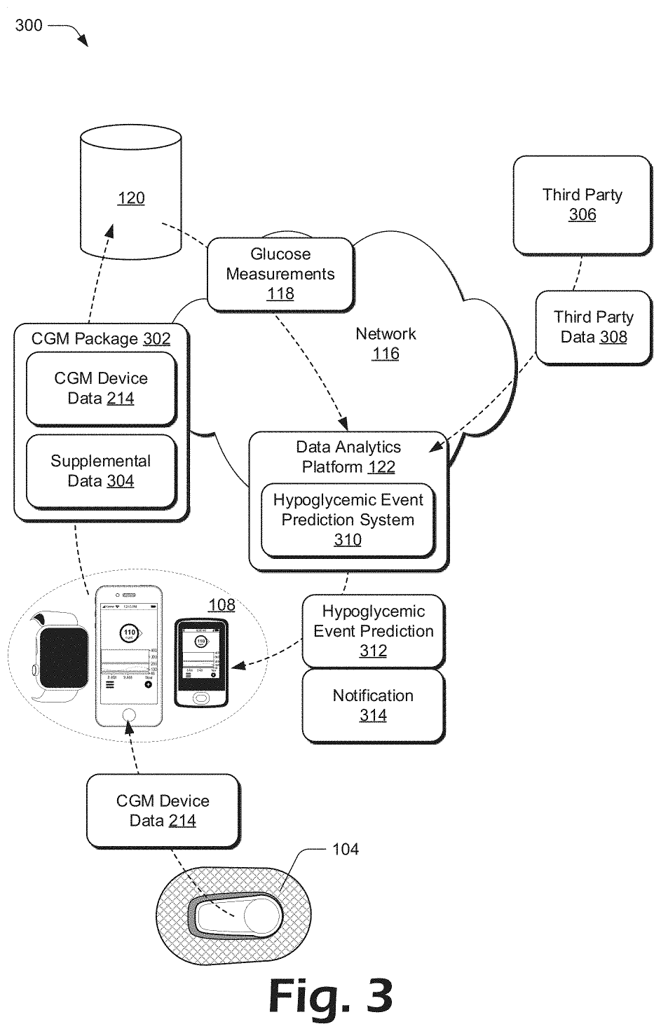

FIG. 3 depicts an example implementation 300 in which CGM device data, including glucose measurements, is routed to different systems in connection with hypoglycemic event prediction using machine learning.

The illustrated example 300 includes from FIG. 1 the CGM system 104 and examples of the computing device 108. The illustrated example 300 also includes the data analytics platform 122 and the storage device 120, which, as discussed above, stores the glucose measurements 118. In this example 300, the CGM system 104 is depicted transmitting the CGM device data 214 to the computing device 108. As discussed above in relation to FIG. 2, the CGM device data 214 includes the glucose measurements 118 along with other data. The CGM system 104 may transmit the CGM device data 214 to the computing device 108 in a variety of ways.

The illustrated example 300 also includes CGM package 302. The CGM package 302 may include the CGM device data 214 (e.g., the glucose measurements 118, the sensor identification 216, and the sensor status 218), supplemental data 304, or portions thereof. In this example 300, the CGM package 302 is depicted being routed from the computing device 108 to the storage device 120 of the CGM platform 112. Broadly speaking, the computing device 108 includes functionality to generate the supplemental data 304 based, at least in part, on the CGM device data 214. The computing device 108 also includes functionality to package the supplemental data 304 together with the CGM device data 214 to form the CGM package 302 and communicate the CGM package 302 to the CGM platform 112 for storage in the storage device 120, e.g., via the network 116. It is to be appreciated, therefore, that the CGM package 302 may include data collected by the CGM system 104 (e.g., the glucose measurements 118 sensed by the sensor 202) as well as supplemental data 304 generated by the computing device 108 that acts as an intermediary between the CGM system 104 and the CGM platform 112, such as a mobile phone or a smart watch of the user.

With respect to the supplemental data 304, the computing device 108 may generate a variety of supplemental data to supplement the CGM device data 214 included in the CGM package 302. In accordance with the described techniques, the supplemental data 304 may describe one or more aspects of a user's context, such that correspondences of the user's context with CGM device data 214 (e.g., the glucose measurements 118) can be identified. By way of example, the supplemental data 304 may describe user interaction with the computing device 108, and include, for instance, data extracted from application logs describing interaction (e.g., selections made, operations performed) for particular applications. The supplemental data 304 may also include clickstream data describing clicks, taps, and presses performed in relation to input/output interfaces of the computing device 108. As another example, the supplemental data 304 may include gaze data describing where a user is looking (e.g., in relation to a display device associated with the computing device 108 or when the user is looking away from the device), voice data describing audible commands and other spoken phrases of the user or other users (e.g., including passively listening to users), device data describing the device (e.g., make, model, operating system and version, camera type, apps the computing device 108 is running), and so on.

The supplemental data 304 may also describe other aspects of a user's context, such as environmental aspects including, for example, a location of the user, a temperature at the location (e.g., outdoor generally, proximate the user using temperature sensing functionality), weather at the location, an altitude of the user, barometric pressure, context information obtained in relation to the user via the IoT 114 (e.g., food the user is eating, a manner in which a user is using sporting equipment, clothes the user is wearing), and so forth. The supplemental data 304 may also describe health-related aspects detected about a user including, for example, steps, heart rate, perspiration, a temperature of the user (e.g., as detected by the computing device 108), and so forth. To the extent that the computing device 108 may include functionality to detect, or otherwise measure, some of the same aspects as the CGM system 104, the data from these two sources may be compared, e.g., for accuracy, fault detection, and so forth. The above-discussed types of the supplemental data 304 are merely examples and the supplemental data 304 may include more, fewer, or different types of data without departing from the spirit or scope of the techniques described herein.

Regardless of how robustly the supplemental data 304 describes a context of a user, the computing device 108 may communicate the CGM packages 302, containing the CGM device data 214 and the supplemental data 304, to the CGM platform 112 for processing at various intervals. In one or more implementations, the computing device 108 may stream the CGM packages 302 to the CGM platform 112 substantially in real-time, e.g., as the CGM system 104 provides the CGM device data 214 continuously to the computing device 108. The computing device 108 may alternately or additionally communicate one or more of the CGM packages 302 to the CGM platform 112 at a predetermined interval, e.g., every second, every 30 seconds, every hour, and so on.

Although not depicted in the illustrated example 300, the CGM platform 112 may process these CGM packages 302 and cause at least some of the CGM device data 214 and the supplemental data 304 to be stored in the storage device 120. From the storage device 120, this data may be provided to, or otherwise accessed by, the data analytics platform 122, e.g., to generate predictions of upcoming glucose levels, as described in more detail below.

In one or more implementations, the data analytics platform 122 may also ingest data from a third party 306 (e.g., a third party service provider) for use in connection with generating predictions of upcoming glucose levels. By way of example, the third party 306 may produce its own, additional data, such as via devices that the third party 306 manufactures and/or deploys, e.g., wearable devices. The illustrated example 300 includes third party data 308, which is shown being communicated from the third party 306 to the data analytics platform 122 and represents this additional data produced by or otherwise communicated from the third party 306.

As mentioned above, the third party 306 may manufacture and/or deploy associated devices. Additionally or alternately, the third party 306 may obtain data through other sources, such as corresponding applications. This data may thus include user-entered data entered via corresponding third party applications, e.g., social networking applications, lifestyle applications, and so forth. Given this, the data produced by the third party 306 may be configured in various ways, including as proprietary data structures, text files, images obtained via mobile devices of users, formats indicative of text entered to exposed fields or dialog boxes, formats indicative of option selections, and so forth.

The third party data 308 may describe various aspects related to one or more services provided by a third party without departing from the spirit or scope of the described techniques. The third party data 308 may include, for instance, application interaction data which describes usage or interaction by users with a particular application provided by the third party 306. Generally, the application interaction data enables the data analytics platform 122 to determine usage, or an amount of usage, of a particular application by users of the user population 110. Such data, for example, may include data extracted from application logs describing user interactions with a particular application, clickstream data describing clicks, taps, and presses performed in relation to input/output interfaces of the application, and so forth. In one or more implementations, the data analytics platform 122 may thus receive the third party data 308 produced or otherwise obtained by the third party 306.

The data analytics platform 122 is illustrated with a hypoglycemic event prediction system 310. In accordance with the described systems, the hypoglycemic event prediction system 310 is configured to generate hypoglycemic event predictions 312 based on the glucose measurements 118. Specifically, the hypoglycemic event prediction system 310 is configured to predict whether or not a hypoglycemic event will occur during an upcoming time interval based on glucose measurements 118 obtained during a previous time interval. For example, the hypoglycemic event prediction system 310 can predict the occurrence (or lack thereof) of a hypoglycemic event during an upcoming night time interval based on glucose measurements 118 obtained during a previous day time interval. As discussed in more detail below, the hypoglycemic event predictions 312 are based on glucose measurements 118 that have been sequenced according to timestamps to form time series glucose measurements, e.g., glucose traces. In one or more implementations, for instance, the hypoglycemic event prediction system 310 may generate hypoglycemic event predictions 312 based on both the glucose measurements 118 and additional data, where the additional data may include one or more portions of the CGM device data 214 additional to the glucose measurements 118, the supplemental data 304, the third party data 308, data from the IoT 114, and so forth. As discussed below, the hypoglycemic event prediction system 310 may generate such hypoglycemic event predictions 312 by using one or more machine learning models. These models may be trained or otherwise built using the glucose measurements 118 and additional data obtained from the user population 110.

Based on the generated hypoglycemic event predictions 312, the data analytics platform 122 may also generate notifications 314. A notification 314, for instance, may alert a user about an upcoming hypoglycemic event during an upcoming night time interval, such that the user is likely to experience a hypoglycemic event during the night absent a mitigating behavior (e.g., eating a particular food or drink). In contrast, the notification 314 may notify the user that the user is unlikely to experience a hypoglycemic event during the night, which may allow the user to go to sleep with confidence that the user is unlikely to experience a hypoglycemic event while sleeping. The notification 314 may also provide support for deciding how to decrease the probability of nighttime hypoglycemic events, such as by recommending the user perform an action (e.g., consume a particular food or drink, download an app to the computing device 108, seek medical attention immediately, decrease insulin dosages, or modify exercise behavior), continue a behavior (e.g., continue eating a certain way or exercising a certain way), change a behavior (e.g., change eating habits or exercise habits, change basal or bolus insulin dosages), and so on.

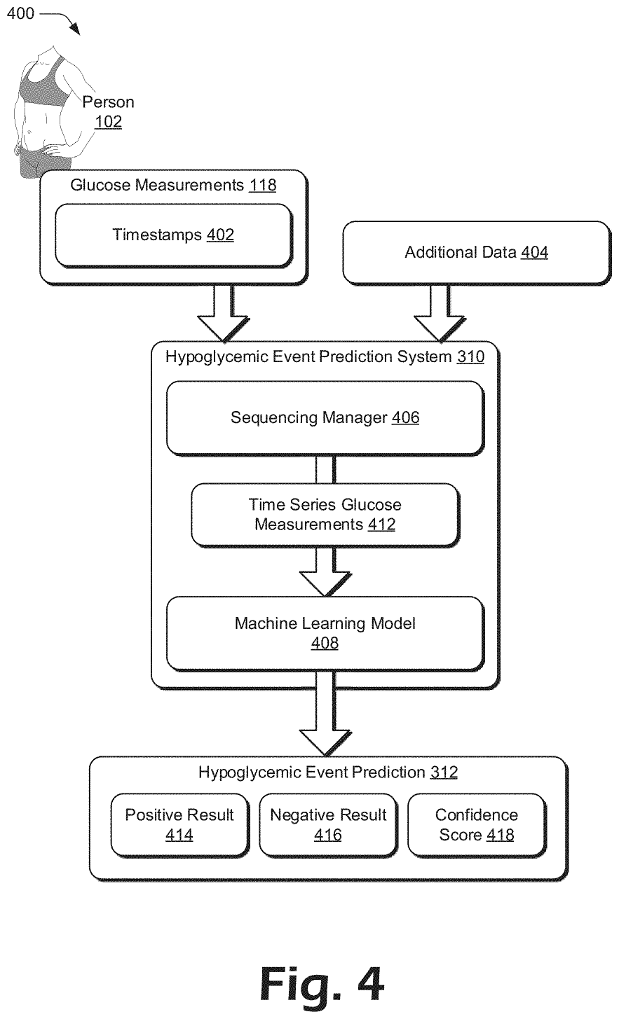

In such scenarios, the hypoglycemic event prediction 312 and/or the notification 314 is communicated from the data analytics platform 122 and output via the computing device 108. In the illustrated example 300, the hypoglycemic event prediction 312 is also illustrated being communicated to the computing device 108. It is to be appreciated that either or both of the hypoglycemic event prediction 312 and the notification 314 may be communicated to the computing device 108. Additionally or alternately the hypoglycemic event prediction 312 and/or the notification 314 may be routed to a decision support platform and/or a validation platform, e.g., before the hypoglycemic event prediction 312 and/or notification 314 are allowed to be delivered to the computing device 108. In the context of generating hypoglycemic event predictions, consider the following discussion of FIG. 4.

FIG. 4 depicts an example implementation 400 of the hypoglycemic event prediction system 310 of FIG. 3 in greater detail to predict whether a hypoglycemic event will occur during an upcoming night time interval using machine learning.

In the illustrated example 400, the hypoglycemic event prediction system 310 is shown obtaining the glucose measurements 118 (e.g., from the storage 120), timestamps 402, and additional data 404. Here, the glucose measurements 118 and the additional data 404 may correspond to the person 102. Additionally, each of the glucose measurements 118 corresponds to one of the timestamps 402. In other words, there may be a one-to-one relationship between glucose measurements 118 and timestamps 402, such that there is a corresponding timestamp 402 for each individual glucose measurement 118. In one or more implementations, the CGM device data 214 may include a glucose measurement 118 and a corresponding timestamp 402. Accordingly, the corresponding timestamp 402 may be associated with the glucose measurement 118 at the CGM system 104 level, e.g., in connection with producing the glucose measurement 118. Regardless of how a timestamp 402 is associated with a glucose measurement 118—or which device associates a timestamp 402 with a glucose measurement 118—each of the glucose measurements 118 has a corresponding timestamp 402.

In this example 400, the hypoglycemic event prediction system 310 is depicted as including sequence manager 406 and a machine learning model 408, which are configured to generate a hypoglycemic event prediction 312 based on the glucose measurements 118, the timestamps 402, and the additional data 404. However, it is to be appreciated that in some implementations the hypoglycemic event prediction system 310 generates the hypoglycemic event prediction using only the time series glucose measurements 412 without the use of additional data of the person 102. Although the hypoglycemic event prediction system 310 is depicted including these two components, it is to be appreciated that the hypoglycemic event prediction system 310 may have more, fewer, and/or different components to generate the hypoglycemic event prediction 312 without departing from the spirit or scope of the described techniques.

Broadly speaking, the sequencing manager 406 is configured to generate time series glucose measurements based on the glucose measurements 118 and the timestamps 302. Although the glucose measurements 118 may generally be received in order, e.g., by the CGM platform 112 from the CGM system 104 and/or the computing device 108, in some instances, one or more of the glucose measurements 118 may not be received in a same order in which the glucose measurements 118 are produced—packets with the glucose measurements 118 may be received out of order. Thus, the order of receipt may not chronologically match the order in which the glucose measurements 118 are produced by the CGM system 104. Alternately or additionally, the communications including one or more of the glucose measurements 118 may be corrupted. Indeed, there may be a variety of reasons why the glucose measurements 118, as obtained by the hypoglycemic event prediction system 310, may not be entirely in time order.

To generate the time series glucose measurements 412, the sequencing manager 406 determines a time-ordered sequence of the glucose measurements 118 according to the respective timestamps 402. The sequencing manger 406 outputs the time-ordered sequence of the glucose measurements 118 as the time series glucose measurements 412. The time series glucose measurements 412 may be configured as or otherwise referred to as a “glucose trace.”

In accordance with described techniques, the sequencing manager 406 generates the time series glucose measurements 412 for a specific time interval. In one or more implementations, the time series glucose measurements 412 correspond to a day time interval of a current day, and are utilized by the machine learning model 408 to predict whether a hypoglycemic event will occur during a night time interval that is subsequent to the day time interval. For example, the time series glucose measurements 412 may be generated by the sequencing manager 406 for a day time interval from 6 AM in the morning to 10 PM at night, in order to predict whether a hypoglycemic event will occur during a night time interval of 10 PM that night to 6 AM the following morning. Thus, unlike conventional systems which extract features from glucose measurements in order to generate predictions, the time series glucose measurements 412 correspond to an entire set of estimated glucose values for person 102 during the day time interval. Notably, the duration and timing of the day time interval may vary based on a variety of factors without departing from the spirit or scope of the described techniques. For example, in some cases the day time interval and night time interval may be customized to the user's sleep schedule. Moreover, in one or more implementations the sequencing manager 406 may generate the time series glucose measurements 412 for a time interval spanning multiple days (e.g., the previous 7 days).

Although in some implementations the machine learning model 408 may be limited to receiving time series glucose measurements 412 (and information about the time series glucose measurements 412) as input, in one or more implementations, the machine learning model 408 also receives, as input, the additional data 404 describing one or more other aspects that impact a person's glucose in the future. The additional data 404 may be correlated in time to the time series of glucose measurements, e.g., based on timestamps associated with the additional data 404. Such additional data 404 may include, by way of example and not limitation, application usage data (e.g., clickstream data describing user interfaces displayed and user interactions with applications via the user interfaces), accelerometer data of a mobile device or smart watch (e.g., indicating that that the person has viewed a user interface of the device and thus has likely seen an alert or information related to a predicted hypoglycemic event), data describing insulin administered (e.g., timing and insulin doses), food consumed (e.g., timing of food consumption, type of food, and/or amount of carbohydrates consumed, activity data from various sensors (e.g., step data, workouts performed, or other data indicative of user activity or exercise), stress, and so forth. Further examples of aspects that may be indicative of a person's glucose in the future include a temperature of the person, an environmental temperature, barometric pressure, and the presence or absence of various health conditions (e.g., pregnancy), to name just a few. Moreover, the additional data 404 may include the supplemental data 304 and/or the third party data 308 described above with reference to FIG. 3.

The machine learning model 408, in this case, is also trained using historical additional data of the user population. Thus, the accuracy of the predictions generated by the machine learning model 408 are increased by utilizing both the time series glucose measurements 412 and the additional data 404 to generate the predictions. For example, the machine learning model 408 can be trained to learn patterns associated with application usage activity, exercise, food consumption, and insulin doses administered, and thus adjust the hypoglycemic event predictions accordingly.

In one or more implementations, the additional data 404 received as input by the machine learning model 408 is associated with an application of the CGM platform 112. For example, an application of the CGM platform 112 may be executed at a user's computing device (e.g., a smartphone or smartwatch) to display the glucose measurements to the user, e.g., in a user interface of an application of the CGM platform. The additional data 404, in this instance, may correspond to screen views or user selections of different controls of the CGM application. Such application usage data enables the machine learning model 408 to learn whether the user is aware of her current glucose condition, which may indicate that the user has taken a mitigating action to correct the glucose condition. For example, if the user looks at the CGM application shortly before going to bed and notices that her blood glucose levels are dropping, she may take a mitigating action to prevent night time hypoglycemia, such as by eating a piece of fruit. This mitigating action may affect the accuracy of the hypoglycemic event prediction. For example, if the system had predicted the occurrence of night time hypoglycemia, then the mitigating action may prevent the occurrence of night time hypoglycemia causing the prediction to be inaccurate. As such, the machine learning model 408 can learn patterns associated with mitigating actions performed by the user, and then adjust the hypoglycemic event predictions accordingly.

In accordance with the described techniques, the time series glucose measurements 412 are provided along with the additional data 404 as input to the machine learning model 408. The machine learning model 408 processes the time series of glucose measurements 412 and the additional data 404 to generate the hypoglycemic event prediction 312. Generally, the hypoglycemic event prediction 312, output by the machine learning model 408, predicts whether a hypoglycemic event will occur for the user during a night time interval, e.g., that is subsequent to the day time interval of the time series glucose measurements 412. Continuing with the example above, if the time series glucose measurements correspond to a day time interval from 6 AM in the morning to 10 PM at night, then the machine learning model 408 can generate the hypoglycemic event prediction 312 for a night time interval that is subsequent to the day time interview, e.g., from 10 PM that night to 6 AM the following morning.

The hypoglycemic event prediction 312 may be output as a positive result 414 if the hypoglycemic event is predicted to occur during the night time interval by the machine learning model 408, or as a negative result 416 if the hypoglycemic event is predicted not to occur during the night time interval by the machine learning model 408. The machine learning model 408 may also generate a confidence score 418 associated with the positive result 414 or negative result 416. Generally, the confidence score 418 indicates a probability that the predicted posited or negative result will occur. As an example, the machine learning model 408 may output the hypoglycemic event prediction 312 as a value between 0 and 1. A threshold may then be applied such that if the value is lower than 0.5 than it indicates a negative result 416 and if the value is above 0.5 it indicates a positive result 414 that the hypoglycemic event will occur. In this example, a positive result 414 with a value of 0.9 will have a higher confidence score 418 than positive result 414 with a value of 0.55.

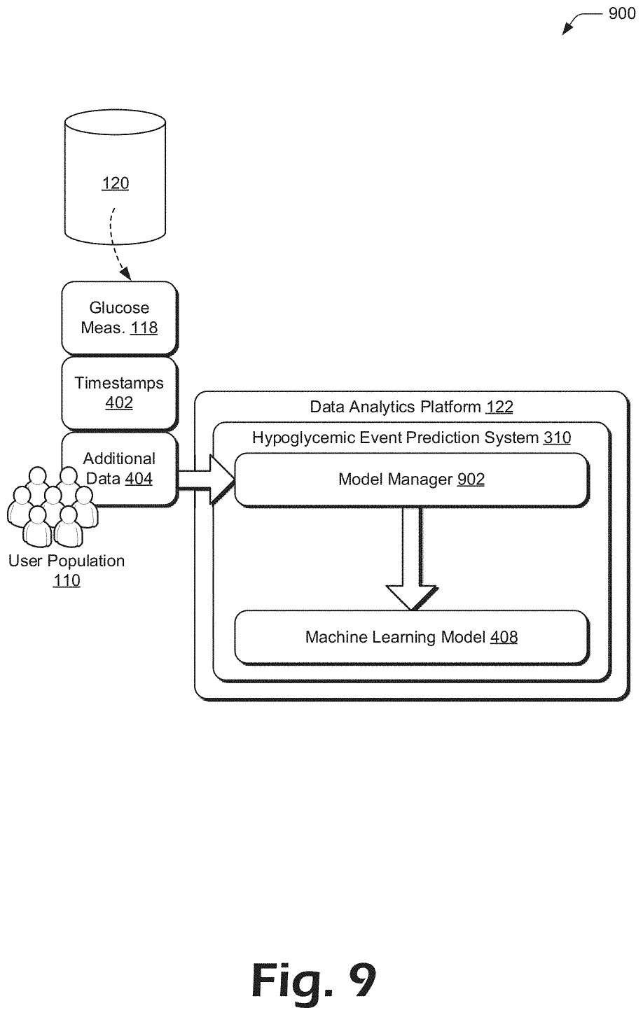

The machine learning model 408 may be trained to output the hypoglycemic event prediction 312 based on the time series glucose measurements 412 and/or the additional data 404. By way of example, the machine learning model 408 may be trained, or an underlying model may be learned, based on one or more training approaches and using labeled historical time series glucose measurements, such as time series glucose measurements 412 generated from the glucose measurements 118 of the user population 110 along with additional data of the user population. Training the machine learning model 408 is discussed in more detail in relation to FIG. 9.

The machine learning model 408 may be implemented in a variety of different ways and utilizing a variety of different types of machine learning models without departing from the spirit or scope of the described techniques. In one or more implementations, the machine learning model 408 is trained to output the hypoglycemic event prediction 312 by classifying the time series glucose measurements 412 as corresponding to the positive result 414 or the negative result 416. For example, the machine learning model 408 learns to classify input streams of estimated glucose values as corresponding to positive result class or a negative result class. The machine learning model 408, in this example, may be implemented as a neural network that obtains, as input, labeled streams of observed glucose values collected over an interval of time. The streams of estimated glucose values are labeled to indicate whether or not a hypoglycemic event occurred later that night. In this way, the machine learning model 408 learns to classify input streams of observed glucose values in order to generate the predicted hypoglycemic event.

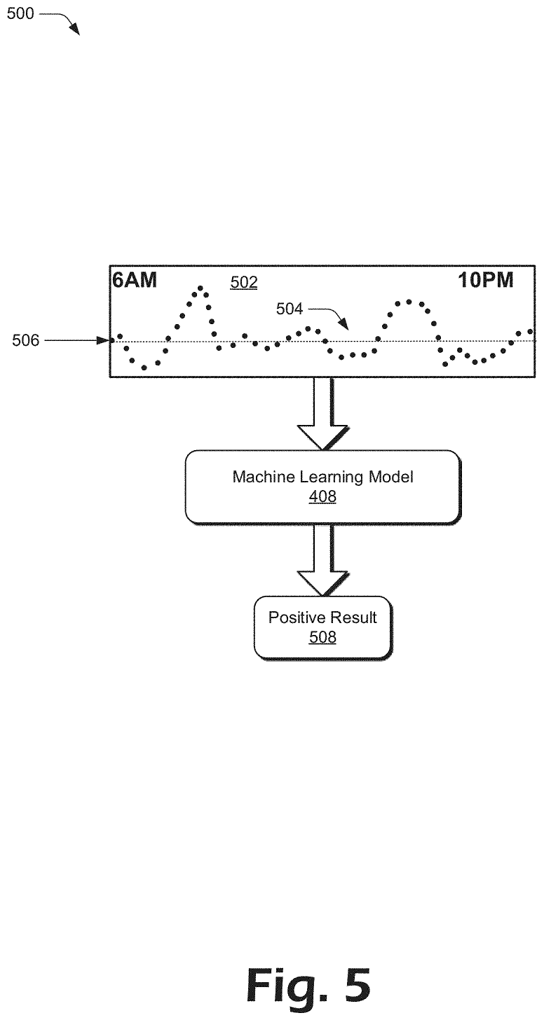

Consider, for example, FIG. 5 which depicts an example implementation 500 in which the machine learning model 408 generates a hypoglycemic event prediction in accordance with one or more implementations. In this example, machine learning model 408 obtains time series glucose measurements 502 which have been observed over a day time interval of 6 AM to 10 PM. The time series glucose measurements 502 include multiple estimated glucose values 504 observed by the CGM system during the time interval. For example, each “point” of the observed estimated glucose values 504 may correspond to an estimated glucose value measured by a CGM system during the day time interval. As such, each observed glucose value 504 includes a respective time stamp, and thus are arranged in a time-ordered sequence. In some cases, the CGM system is configured to generate glucose values 504 at predetermined time intervals, such as every 5 minutes. In this example, a day time interval of 16 hours (e.g., from 6 AM to 10 PM) would include 192 distinct glucose values 504. The time series glucose measurements 502 are shown with a hypoglycemic threshold 506 corresponding to a blood glucose level that is considered hypoglycemic if the user's blood glucose levels are below this range. For example, the hypoglycemic threshold 506 may correspond to a value of 70 mg/dl in this example, but can be set to other values such as 60 mg/dl. Based on the time series glucose measurements 502, the machine learning model 408 generates a hypoglycemic event prediction 508, which in this example is a positive result. In other words, based on the input time series glucose measurements 502 for the day time interval, the machine learning model predicts that a hypoglycemic event will occur in the upcoming night time interval.

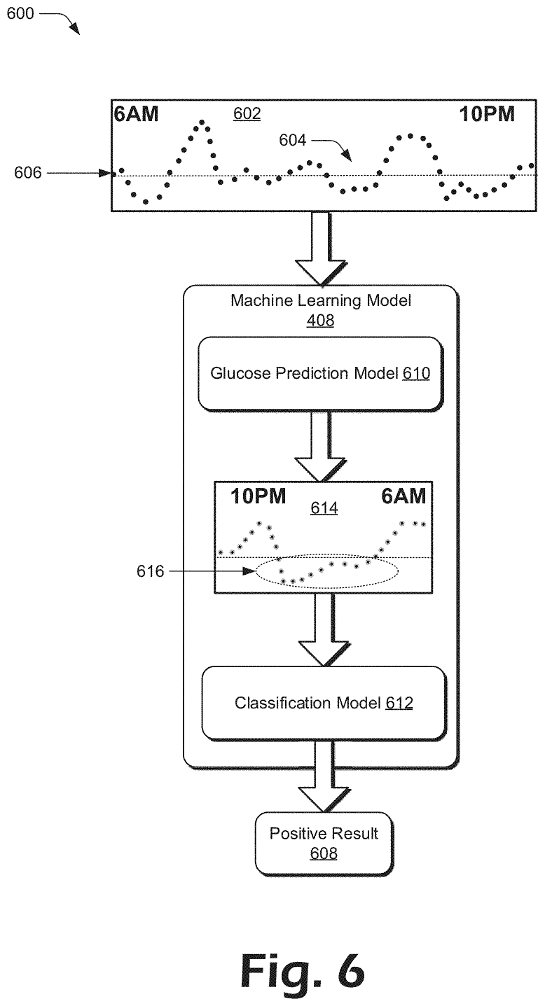

In one or more implementations, the machine learning model 408 is trained to first predict upcoming glucose measurements for the night time interval based on time series glucose measurements observed during the day time interval, and then to generate the hypoglycemic event prediction 312 based on the predicted upcoming glucose measurements. Consider, for example, FIG. 6 which depicts an additional example implementation 600 in which the machine learning model 408 generates a hypoglycemic event prediction in accordance with one or more implementations. Similar to example 500, the machine learning model 408 obtains time series glucose measurements 602 which have been observed over a day time interval of 6 AM to 10 PM. The time series glucose measurements 602 include multiple estimated glucose values 604 observed by the CGM system during the day time interval. The time series glucose measurements 602 are shown with a hypoglycemic threshold 606 corresponding to a blood glucose level that is considered hypoglycemic if the user's blood glucose levels are below this range.

Based on the time series glucose measurements 602, the machine learning model 408 generates a hypoglycemic event prediction 608, which in this example is a positive result. Unlike example 500, however, the machine learning model 408 is depicted as including a glucose prediction model 610 and a classification model 612. Generally, the glucose prediction model 610 is configured to generate and output predicted upcoming glucose measurements 614 based on the time series glucose measurements 602. By way of example, the glucose prediction model 610 may be trained, or an underlying model may be learned, based on one or more training approaches and using historical time series glucose measurements, such as time series glucose measurements generated from the glucose measurements 118 of the user population 110.

Notably, the predicted upcoming glucose measurements 614 correspond to predicted glucose measurements over the upcoming night time interval, while the time series glucose measurements are a trace of glucose measurements that have been observed by a CGM system, such as by the CGM system 104 worn by the person 102 over the day time interval. Thus, glucose measurements observed in this way contrast with glucose measurements predicted, e.g., by the glucose prediction model 610. In this example, the time series glucose measurements 602 correspond to a trace of the glucose measurements 118 observed for the person 102 over a day time interval (e.g., from 6 AM to 10 PM) and the predicted upcoming glucose measurements 614 may be configured as predicted glucose traces for an upcoming night time interval corresponding to the next 8 hours of the night (e.g., from 10 PM to 6 AM).

The classification model 612 receives predicted upcoming glucose measurements 614, and outputs the hypoglycemic event prediction 608. In this case, the hypoglycemic event prediction 608 is based on the predicted upcoming glucose measurements 614. Notably, the upcoming predicted glucose measurements 614 include multiple glucose values 616 below the hypoglycemic threshold 606. In this example, therefore, the classification model 612 generates the prediction that the hypoglycemic event will occur during the night time interval based on the predicted glucose measurements 616 which are below the hypoglycemic threshold 606.

The classification model 612 may be configured to predict the occurrence of the hypoglycemic event based on a variety of different factors. In some cases, a positive result is predicted by the classification model 612 if there are four or more consecutive predicted glucose values in the night time interval which are below the hypoglycemic threshold 606. However, the hypoglycemic threshold and the number of glucose values below the threshold may vary without departing from the spirit and scope of the described techniques.

Notably, the glucose prediction model 610 may generate the predicted upcoming time series glucose measurements 614 in a variety of different ways. In one or more implementations, the glucose prediction model 610 may be implemented as a vector output model or an encoder-decoder model that is trained to predict the entire sequence of glucose measurements during the night time interval based on the input sequence of glucose measurements of the day time interval. In other words, the input to the glucose prediction model 610 is a single day or multi-day sequence of glucose values, and the output of the glucose prediction model 610 is a sequence of predicted glucose values for the entire night time interval. The positive or negative hypoglycemia result classification is then applied to the entire predicted glucose value sequence for the night time interval.

Alternately, the glucose prediction model 610 may be trained to predict a single glucose value for the night time interval, and then the process can be iterated to predict the entire glucose value sequence for the night time interval. In other words, the input to the glucose prediction model 610 is a single day or multi-day sequence of glucose values, and the output of the glucose prediction model 610 is a single predicted glucose value. Then, the observed glucose measurements, along with the single predicted glucose value are input back into the glucose prediction model 610 in order to generate a next predicted glucose value. This process is then repeated for multiple iterations in order to predict the entire nighttime sequence of glucose values. In this implementation, the glucose predication model 610 may be configured as a non-linear model or an ensemble of models that includes one or more non-linear models. Non-linear machine learning models may include, for instance, neural networks (e.g., recurrent neural networks such as long-short term memory (LSTM)), state machines, Markov chains, Monte Carlo methods, and particle filters, to name just a few. It is to be appreciated that the glucose prediction model 610 may be configured as or otherwise include one or more different types of machine learning models without departing from the spirit or scope of the described techniques.

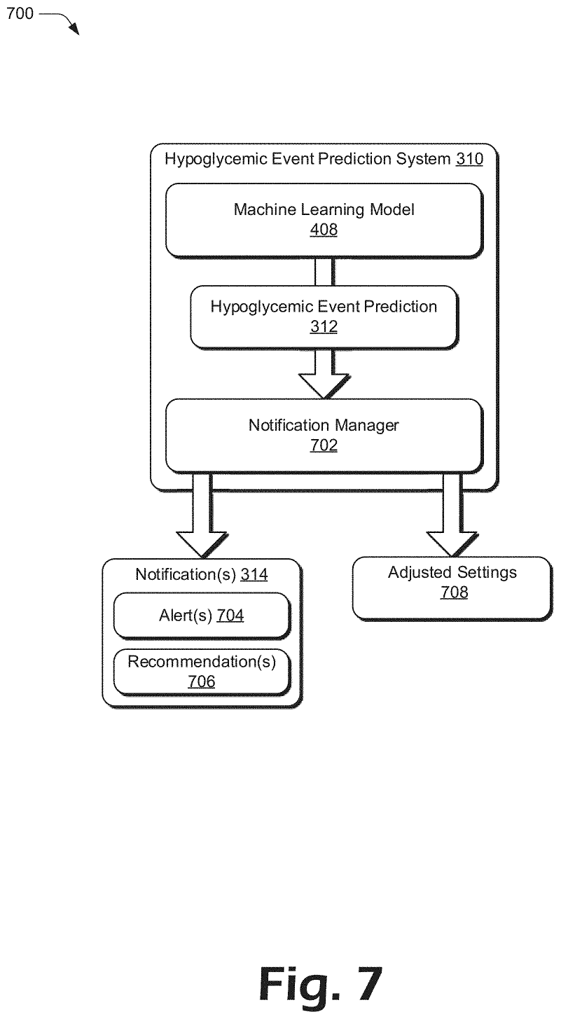

FIG. 7 depicts an example implementation 700 of the hypoglycemic event prediction system 310 of FIG. 3 in greater detail to output notifications 314 based on a hypoglycemic event prediction 312.

In the illustrated example 700, the hypoglycemic event prediction system 310 is depicted as including a notification manager 702 that obtains the hypoglycemic event prediction 312 from the machine learning model 408. The notification manager 702 generates and delivers notifications 314 based on the hypoglycemic event prediction 312 output by the machine learning model 408. The notification 314 may include an alert 704 that informs person 102 of the likelihood the person will experience a hypoglycemic event during the upcoming night. For example, the alert may indicate that the user is predicted to experience a hypoglycemic event during the upcoming night if the hypoglycemic event prediction 312 corresponds to the positive result 414. In contrast, the alert may indicate that the user is not predicted to experience the hypoglycemic event during the upcoming night if the hypoglycemic event prediction 312 corresponds to the negative result 416.

The notification 314 may also include one or more recommendations 706. For example, if the machine learning model 408 predicts that the person 102 is likely to experience hypoglycemia during the night, then the notification manager 702 may output one or more recommendations 706 for mitigating the hypoglycemia, such as to drink a glass of juice before sleep, eat a piece of fruit before sleep, set an alarm for a certain time to wake up and drink juice or eat fruit, and so forth. On the other hand, if the machine learning model 408 predicts that the user is not likely to experience hypoglycemia over the upcoming predetermined time period, then the notification manager 702 can output the a notification indicating that this is the case and/or that no mitigating actions need to be taken.

In one or more implementations, the notification 314 may also include a visual representation of the confidence score 418 to inform the user of the accuracy of the prediction. For example, if the machine learning model 408 predicts the occurrence of a hypoglycemic event during the night with 90% confidence, then the notification 314 may visually indicate that this confidence level to the user as part of the alert 704. Alternately, if the machine learning model 408 predicts that the user will not experience an episode of hypoglycemia during the night with 90% confidence, then the notification 314 may visually indicate this confidence level to the user as part of the alert 704.

In one or more implementations, the alert 704 and/or the recommendation 706 generated by the notification manager 702 may be based, at least in part, on the confidence score 418. The notification manager 702, for example, may provide different alerts 704, recommendations 706, or other messaging based in part on the confidence level associated with the prediction. For example, if the machine learning model predicts with a high confidence that the user will have or not have a hypoglycemic event during the night, then the notification manager 702 may output this prediction to the user. However, in cases where the confidence level is lower, the notification manger may adjust the messaging output to the user, such as by cautioning the user that the prediction is made with lower confidence, asking the user to generate the prediction again at a later time period, or notifying the user that the system is unable to generate a prediction at this time.

In one or more implementations, the hypoglycemic event prediction system 310 can generate multiple hypoglycemic event predictions 312 at different times in order to increase the accuracy of the hypoglycemic event predictions 312. For example, as described above, the hypoglycemic event prediction system 310 can generate an initial hypoglycemic event prediction 312, that predicts whether the hypoglycemic event will occur during the night time interval that is subsequent to the day time interval, by processing the time series of glucose measurements 412 using the machine learning model 408. This initial prediction, for example, may be generated by the hypoglycemic event prediction system 310 an hour before the user plans to go to sleep. Then, after the initial prediction is generated, the hypoglycemic event prediction system 312 may receive an additional time series of glucose measurements 412. In other words, the additional time series of glucose measurements 412 can be provided by the CGM system worn by the user during a subsequent period of time that occurs after outputting the initial hypoglycemic event prediction 312. Then, the hypoglycemic event prediction system 310 can generate an updated hypoglycemic event prediction 312, that predicts whether the hypoglycemic event will occur during the night time interval, by processing the additional time series of glucose measurements 412 using the machine learning model 408. The updated prediction, for example, may be generated by the hypoglycemic event prediction system 310 an hour after the initial prediction is generated, and then right before the user plans to go to sleep.

Notably, the updated hypoglycemic event prediction 312 can be generated using the machine learning model 408 in order to confirm the accuracy of an initial prediction that the user will not experience a hypoglycemic event during the night time interval (e.g., the negative result 416), or to confirm that a mitigating action taken by the user to mitigate a predicted hypoglycemic event (e.g., a positive result 414) was sufficient to change the prediction to a negative result 416. By way of example, if the hypoglycemic event prediction system 310 executes a first instance of the machine learning model 408 an hour before the user's bedtime that predicts that the user will not experience a hypoglycemic event, then the hypoglycemic event prediction system 310 can execute a second instance of the machine learning model 408 a fixed amount of time later (e.g., just prior to the user going to sleep) in order to confirm that the initial prediction was accurate. In this example, if both the initial prediction and the updated prediction generated by the hypoglycemic event prediction system 310 comprises the negative result 416, e.g., that the user will not experience a hypoglycemic event during the night, then the accuracy of the prediction is increased.

As another example, consider that the hypoglycemic event prediction system 310 executes a first instance of the machine learning model 408 an hour before the user's bedtime that predicts that the user will experience a hypoglycemic event during the night time interval (e.g., the positive result 414) along with a recommendation to mitigate the hypoglycemic event, e.g., a recommendation to drink a glass of juice or eat a piece of fruit. In this scenario, the hypoglycemic event prediction system 310 can execute a second instance of the machine learning model a fixed amount of time later in order to confirm that the user took the recommended action that that this action was sufficient to mitigate the predicted hypoglycemic event, e.g., that the hypoglycemic event prediction 312 now predicts that the user will not experience the hypoglycemic event during the night. In this example, therefore, the updated prediction confirms that recommended action was sufficient to prevent the user from experiencing the hypoglycemic event. Outputting the updated prediction, in this scenario, gives the user peace of mind before the user goes to sleep that the recommended action will prevent the hypoglycemic event from occurring during the night.

Moreover, in cases where the first instance of the machine learning model predicts that the user will not experience a hypoglycemic event during the night time interval, the hypoglycemic event prediction system 310 can generate a recommendation that the user not take any intervening action (e.g., dosing insulin or consuming carbs) prior to the execution of the second “confirmation” machine learning model 408. In this scenario, the second machine learning model 408 may be more heavily weighted towards the new data (e.g., the glucose measurements 118 and/or additional data 404 obtained after the first hypoglycemic event prediction 312 is generated) in order to confirm the initial prediction. Notably, prompting the user to abstain from taking a mitigating action in these scenarios may further increase the accuracy of the hypoglycemic event predictions 312 generated by the hypoglycemic event prediction system.

In one or more implementations, the CGM platform may adjust various settings for the night time interval based on hypoglycemic event prediction 312. In example 700, the notification manager 702 is depicted as generating adjusted settings 708 based on the hypoglycemic event prediction. In one or more implementations, the adjusted settings 708 correspond to adjusting the glucose alert settings for the night time interval if a negative result is predicted. For example, a threshold for a low glucose alert may be adjusted by raising the threshold during the night time interval when the machine learning model 408 predicts that the user will not experience an episode of hypoglycemia. This has the effect of triggering a low glucose alert earlier than usual in order to give person 102 more time to mitigate their low glucose level in the event that a hypoglycemic event is experienced after the system predicts that a hypoglycemic event will not occur. The adjusted settings 708 may also override any customized alert settings that have been modified by person 102. As such, the system takes an action even if the prediction is negative.

Alternatively or in addition to adjusting the glucose alert settings to give the user advanced warning in the event a hypoglycemic event is experienced during the night after the system predicts that a hypoglycemic event will not occur, the hypoglycemic event prediction system 310 may be implemented to generate additional hypoglycemic event predictions 312 during the night time interval (e.g., while the user is sleeping) using the machine learning model 408. Notably, the additional predictions generated during the night time interval may confirm the initial prediction that the user will not experience a hypoglycemic event. In this case, no additional actions may be taken by the hypoglycemic event prediction system. If, on the other hand, the hypoglycemic event prediction system 310 initially predicts that the hypoglycemic event will not occur during the night time interval, but then generates an additional prediction during the night time interval that predicts that the hypoglycemic event will now occur due to changing conditions, the hypoglycemic event prediction system 310 can then generate an alert that causes the user to wake up and take a mitigating action.

Notably, the generation of a hypoglycemic event while the user is sleeping—and after the hypoglycemic event prediction system 310 had originally predicted that the user will not experience hypoglycemia during the night—may be disruptive to the user's sleep. As such, the hypoglycemic event prediction system 310 can be configured to generate the additional hypoglycemic event predictions 312 during a window of time at the beginning of the night time interval, e.g., a 30 or 60 minute window of time that begins after the user goes to bed. For example, if the user goes to sleep at 9 pm, the hypoglycemic event prediction system 310 can generate the additional prediction based on glucose measurements 118 captured between 9 pm and 10 pm. In this way, if the hypoglycemic event prediction system 310 predicts that the hypoglycemic event will occur, the user will be notified early during their planned sleep window, as opposed to being awakened later in the night when the user may be in a deep sleep.

Notably, these additional safeguards (e.g., adjusting the alert threshold and/or generating additional predictions during the night time interval) may serve to mitigate the risk associated with a false prediction that hypoglycemia will not occur during the night by providing additional layers of safety protocols which the user can rely on in the event that the conditions evolve rapidly or unexpectedly. Moreover, these additional protections may enable the user to have more trust in the predictions generated by the CGM platform, thereby increasing their quality of life during sleeping hours by reducing cognitive burden.

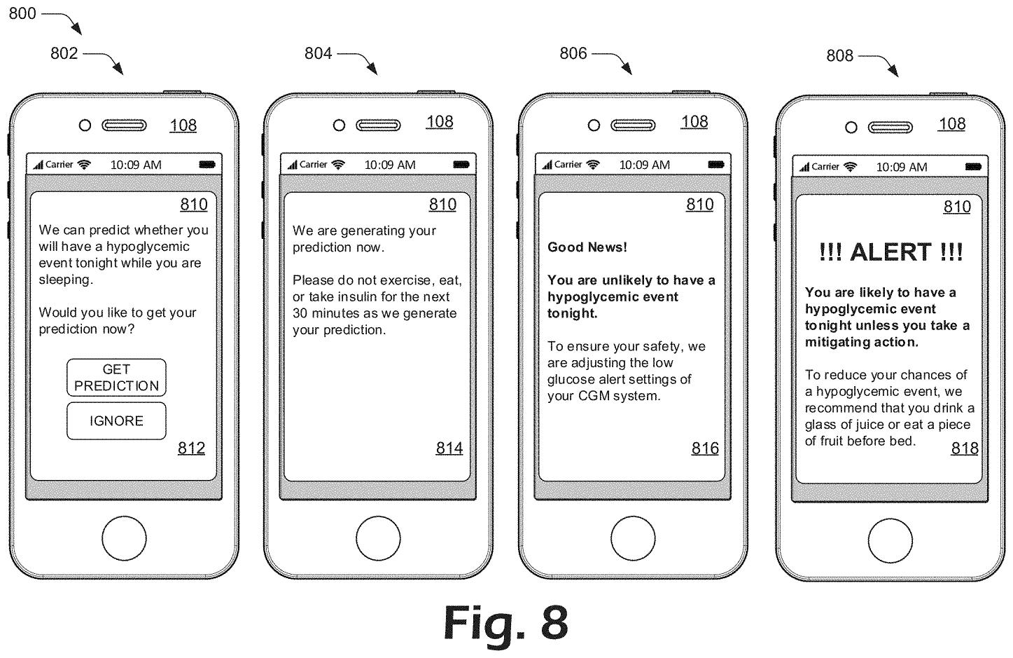

In the context of outputting notifications 314 to the user, consider FIG. 8 which depicts example implementations 800 of user interfaces displayed for notifying a user based on predictions of hypoglycemic events occurring during a night time interval. In particular, the example implementations 800 include the computing device 108 depicted in a user request scenario 802, a prediction generation scenario 804, a negative result scenario 806, and a positive result scenario 808.

In each of scenarios 802, 804, 806, and 808, the computing device 108 displays a user interface 810. The user interface 810 may correspond to an interface of an application, e.g., an interface of the CGM platform 112. Alternately or additionally, the user interface 810 may correspond to a notification “center”, such as a lock screen or other operating-level screen.

The hypoglycemic event prediction system 310 can generate and output hypoglycemic event predictions to the user automatically, or in response to a user request. This decision may be user configurable, as some users may prefer to receive these predictions automatically (e.g., at a set time period), while other users may prefer to only receive these predictions when requested, such as by requesting the prediction before the user goes to sleep. The request scenario 802 depicts an example scenario in which the prediction system generates the prediction in response to a user request. In the request scenario 802, the user interface 810 displays a request control 812 which asks the user whether they would like to receive a hypoglycemic event prediction for the upcoming night. If the user selects the “get prediction” control, the hypoglycemic event prediction system 310 generates the hypoglycemic event prediction 312, as described throughout. Alternately, the user can select ignore if she does not want to receive the prediction.

In one or more implementations, the machine learning model 408 may increase the accuracy of the hypoglycemic event prediction 312 if the model knows that the user is not performing any actions which may affect the user's blood glucose levels, such as eating, exercising, or taking insulin. In some cases, therefore, the system may output a request that the user refrain from behavior that affects glucose levels for a certain period of time while the prediction is being generated. The prediction generation scenario 804 shows the user interface 810 displaying a notification 814 informing the user that the hypoglycemic event prediction is being generated, while also asking the user to refrain from exercising, eating, or dosing insulin for the next 30 minutes while the prediction is being generated.

Regardless of whether the prediction is generated automatically or in response to a user request, the hypoglycemic event prediction system 310 outputs notifications 314 which inform the user of the positive or negative hypoglycemic event prediction. In the negative result scenario 806, the user interface 810 displays negative result alert notification 816 via a display device of the computing device 108. This notification 816 informs the user the she is unlikely to have a hypoglycemic event tonight. In accordance with the described techniques, this notification 816 is based on the hypoglycemic event prediction 312 generated by the hypoglycemic event prediction system 310, which in this case predicts that a hypoglycemic event is unlikely to occur in the night time interval. As discussed above, the system settings of the CGM platform 112 may be adjusted in cases where a negative result is detected and output to the user. Thus, in this example, notification 816 also informs the user that the low glucose alert settings are being adjusted to ensure the safety of the user.

Conversely, in the positive result scenario 808, the user interface 810 displays a positive result alert notification 818 via a display device of the computing device 108. This notification 818 informs the user the she is likely to have a hypoglycemic event tonight. In accordance with the described techniques, this notification 818 is based on the hypoglycemic event prediction 312 generated by the hypoglycemic event prediction system 310, which in this case predicts that a hypoglycemic event is likely to occur in the night time interval. Moreover, the positive result alert notification 818, in this example, provides a recommendation of a suggested action for the user to take in order to mitigate the probability that the hypoglycemic event will occur. In this example, the system recommends that the user drink a glass of juice or eat a piece of fruit before bed.

Although notifications to a user are shown, it is to be appreciated that in one or more implementations, notifications generated based on the hypoglycemic event prediction for the night time interval may alternately or additionally be communicated to other entities, such as a health care provider of the person 102 (e.g., a doctor), a caregiver of the person 102 (e.g., a parent or a child), and so forth. Further, it is to be appreciated that a variety of other services in addition or alternate to notification may be provided based on the hypoglycemic event prediction without departing from the spirit or scope of the described techniques.

FIG. 9 depicts an example implementation 900 of the hypoglycemic event prediction system 310 in greater detail in which a machine learning model is trained to predict whether a hypoglycemic event will occur during a night time interval. As in FIG. 3, the hypoglycemic event prediction system 310 is included as part of the data analytics platform 122, although in other scenarios the hypoglycemic event prediction system 310 may also or alternately be, in part or entirely, included in other devices such as the computing device 108.

In the illustrated example 900, the hypoglycemic event prediction system 310 includes model manager 902, which manages the machine learning model 408, which as mentioned above may be configured as, or includes, one or more machine learning models such as a recurrent neural network, a convolutional neural network, and the like. It is to be appreciated that the machine learning model 408 may be configured as or include other types of machine learning models without departing from the spirit or scope of the described techniques. These different machine learning models may be built or trained (or the model otherwise learned), respectively, using different algorithms due, at least in part, to different architectures. Accordingly, it is to be appreciated that the following discussion of the model manager 902's functionality is applicable to a variety of machine learning models. For explanatory purposes, however, the functionality of the model manager 902 will be described generally in relation to training a neural network.

Broadly speaking, the model manager 902 is configured to manage the machine learning models, including the machine learning model 408. This model management includes, for example, building the machine learning model 408, training the machine learning model 408, updating this model, and so on. In one or more implementations, updating this model may include transfer learning to personalize the machine learning model 408—to personalize it from a state as trained with training data of the user population 110 to an updated state trained with additional training data or (update data) describing one or more aspects of the person 102 and/or describing one or more aspects of a subset of the user population 110 determined similar to the person. Specifically, the model manager 902 is configured to carry out model management using, at least in part, the wealth of data maintained in the storage device 120 of the CGM platform 112. As illustrated, this data includes the glucose measurements 118, timestamps 402, and additional data 404 of the user population 110. Said another way, the model manager 902 builds the machine learning model 408, trains the machine learning model 408 (or otherwise learns an underlying model), and updates this model using the glucose measurements 118, the timestamps 402, and the additional data 404 of the user population 110.

Unlike conventional systems, the CGM platform 112 stores (e.g., in the storage device 120) or otherwise has access to glucose measurements 118 obtained using the CGM system 104 for hundreds of thousands of users of the user population 110 (e.g., 500,000 or more). Moreover, these measurements are taken by sensors of the CGM system 104 at a continuous rate. As a result, the glucose measurements 118 available to the model manager 902, for model building and training, number in the millions, or even billions. With such a robust amount of data, the model manager 902 can build and train the machine learning model 408 to accurately predict whether a hypoglycemic event will occur for a person during an upcoming night time interval based on patterns in their observed glucose measurements.

Absent the robustness of the CGM platform 112's glucose measurements 118, conventional systems simply cannot build or train models to cover state spaces in a manner that suitably represents how patterns indicate future glucose levels. Failure to suitably cover these state spaces can result in hypoglycemic event predictions that are inaccurate, which can lead to results ranging from user annoyance (e.g., providing notifications indicated that a predicted hypoglycemic event will occur that does not in fact take place) to life or death situations (e.g., unsafe conditions resulting from the occurrence of hypoglycemic events during the night when none are predicted). Given the gravity of generating inaccurate and untimely predictions, it is important to build the machine learning model 408 using an amount of glucose measurements 118 that is robust against rare events.

In one or more implementations, the model manager 902 builds the machine learning model 408 by generating training data. Initially, generating the training data includes forming training time series of glucose measurements from the glucose measurements 118 and the corresponding timestamps 402 of the user population 110. The model manager 902 may leverage the functionality of the sequencing manager 406 to form those training time series, for instance, in a similar manner as discussed in detail above in relation to forming the time series glucose measurements 412. The model manager 902 may be further implemented to generate the training time series glucose measurements for a specific time interval. In one or more implementations, the model manager 902 generates the training data to include the time series glucose measurements for a 24-hour period of time corresponding to day.

For each of the training time series (e.g., corresponding to a 24-hour period of time), the model manager 902 may then identify a first portion of the training time series corresponding to a day time interval and second portion of the training time series corresponding to a night time interval. The model manager 902 may then generate, for each instance of training data, a classification label that defines the instance of training data as hypoglycemic positive or hypoglycemic negative based on the time series glucose measurements of the night time interval. For example, instances of training data with a certain number of glucose values below a defined hypoglycemic threshold (e.g., four consecutive glucose value below 70 mg/dl) are classified as hypoglycemic positive, whereas instances of training data without said number of glucose values below the defined hypoglycemic threshold are classified as hypoglycemic positive. The classification labels, therefore, serve as a ground truth for comparison to the model's output during training.

To demonstrate, consider again the example in which the machine learning model 408 receives 24-hours of time series glucose measurements (e.g., 24-hour glucose traces), and identifies the first 16 hours as corresponding to a day time interval and the remaining 8 hours as corresponding to a night time interval. By way of example, a particular training time series may span from 6:00:00 AM on Apr. 15, 2020 to 6:00:00 AM on Apr. 16, 2020. In this case, the model manager 902 may identify a 16-hour portion corresponding to a day time interval, such as from 6:00:00 AM on Apr. 15, 2020 through 10:00:00 PM on Apr. 16, 2020, and an 8-hour portion that spans from 10:01:00 PM on Apr. 15, 2020 through 6:00:00 AM on Apr. 16, 2020. Then, the model manager 902 may then generate, for each instance of training data, a classification label that defines the instance of training data as hypoglycemic positive or hypoglycemic negative based on the time series glucose measurements of the night time interval. Accordingly, once built, the machine learning model 408 is configured to generate a hypoglycemic event prediction for the night time interval based on the glucose traces for the day time interval.

The model manager 902 uses the segmented instances of training data along with the respective classification labels which define the training data as hypoglycemic positive or negative to train the machine learning model 408. In the context of training, the model manager 902 may train the machine learning model 408 by providing an instance of data, corresponding to a day time interval, from the set of training data to the machine learning model 408. Responsive to this, the machine learning model 408 generates a hypoglycemic event prediction for the night time interval, such as by predicting that a hypoglycemic event will occur or not occur during the night time interval. The model manager 902 obtains this training prediction from the machine learning model 408 as output and compares the training prediction to the expected output portion that corresponds to the classification label for the instance of training data. Based on this comparison, the model manager 902 adjusts internal weights of the machine learning model 408 so that the machine learning model can substantially reproduce the expected classification label (e.g., whether night time hypoglycemia will occur) when glucose traces for a day time interval are provided as input in the future.

In one or more implementations, the model manager 902 trains the machine learning model 408 to predict the classification label based on the first portion of training data corresponding to the day time interval. In this case, the machine learning model learns to predict a classification label based on the glucose traces for the day time interval. Alternately, the model manager 902 can train the machine learning model to first predict glucose measurements for the night time interval based on the first portion of training data corresponding to the day time interval, and then generates the hypoglycemic event prediction based on the predicted glucose measurements for the night time interval. In other words, the hypoglycemic event prediction is based on whether there are a predetermined number of predicted glucose values for the night time interval that are below the hypoglycemic threshold. In this instance, the machine learning model can learn to predict the upcoming glucose measurements for a night time interval in stepped implementations (e.g., LSTM) or predicting an entire night time interval in non-stepped implementations (e.g., other types of neural networks).

This process of inputting instances of the training data into the machine learning model 408, receiving training predictions from the machine learning model 408, comparing the training predictions to the expected classification labels (observed) that correspond to the occurrence of a hypoglycemic event during the input night time interval of the training data (e.g., using a cost function), and adjusting internal weights of the machine learning model 408 based on these comparisons, can be repeated for hundreds, thousands, or even millions of iterations—using an instance of training data per iteration.

The model manager 902 may perform such iterations until the machine learning model 408 is able to generate predictions that consistently and substantially match the expected output portions. The capability of a machine learning model to consistently generate predictions that substantially match expected output portions may be referred to as “convergence.” Given this, it may be said that the model manger 902 trains the machine learning model 408 until it “converges” on a solution, e.g., the internal weights of the model have been suitably adjusted due to training iterations so that the model consistently generates predictions that substantially match the expected classification labels.

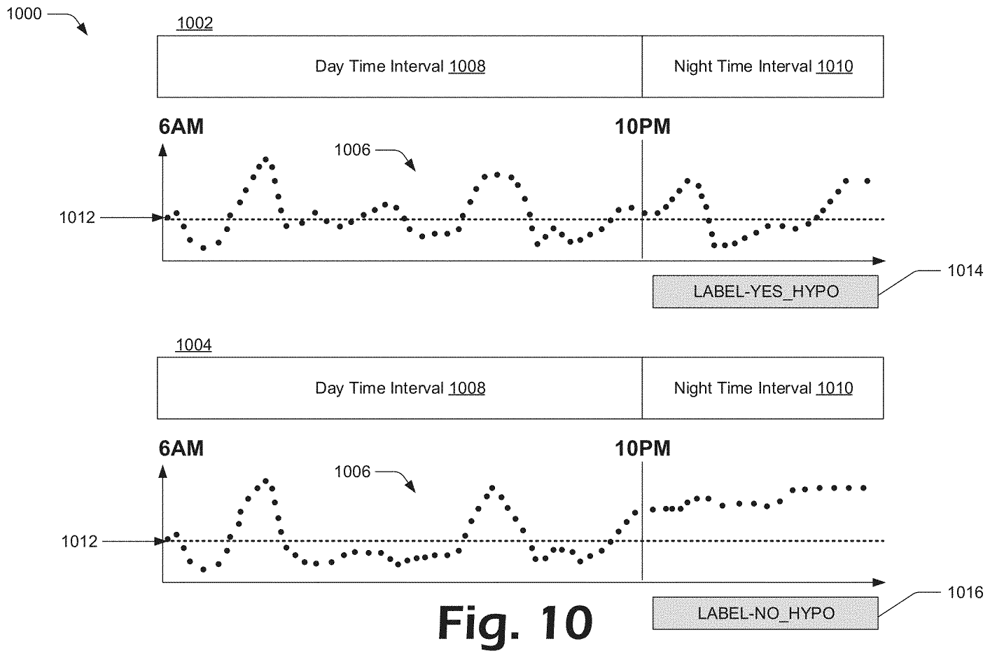

In the context of generating training data, consider FIG. 10 which illustrates an example implementation 1000 of training data generated by the model manager 902 for training the machine learning model. Example 1000 includes example instances of training data 1002 and 1004, each of which contain time series glucose measurements for a user of the user population 110. In this example, instances of training data 1002 and 1004 each include a sequence of glucose measurements 1006 for an entire 24-hour period of time corresponding to a day. In the event that the glucose measurements are taken every 5 minutes by the CGM system, for example, an entire day of training data will include 288 estimated glucose values. In this example, the instances of training data 1002 and 1004 corresponds to a 24-hour period of time from 6:00 AM on a first day to 6:00 AM the following day. Of course, the start and end times for the instances of training data may vary without departing from the spirit or scope of the described techniques.

As discussed above, the model manager 902 is configured to segment each instance of training data into a day time interval and a night time interval. In FIG. 10, for example, the instances of training data 1002 and 1004 have been segmented into a day time interval 1008, which includes glucose measurements 1006 from 6:00 AM to 10:00 PM, and a night time interval 1010 which includes glucose measurements 1006 from 10:01 PM to 6:00 AM the following day. The model manager 902 has classified each instance of training data based on the occurrence, or lack thereof, of hypoglycemia during the night time interval. For example, instances of training data which includes four consecutive estimated glucose values below a hypoglycemic threshold 1012 (e.g., 70 mg/dl) may be classified as hypoglycemic positive, whereas training data that does not have four estimated glucose values below the hypoglycemic threshold 1012 may be classified as hypoglycemic negative. In FIG. 10, for example, training data 1002 has been assigned a “YES_HYPO” label 1014 by the model manager 902 to classify the training data 1002 as hypoglycemic positive because of the occurrence of multiple glucose measurements 1006 below a hypoglycemic threshold 1012 in the night time interval 1010. Similarly, training data 1004 has been assigned a “NO_HYPO” label 1016 by the model manager 902 to classify the training data 1004 as hypoglycemic negative because there are not four consecutive occurrences of glucose measurements 1006 below a hypoglycemic threshold 1012 in the night time interval 1010. In one or more implementations, the model manager 902 may be further configured to classify training data to indicate multiple hypoglycemic events for a given night in cases in which a hypoglycemic event is interrupted by at least one estimated glucose value above the hypoglycemic threshold. It is to be appreciated that the criteria for classifying an instance of training data as hypoglycemic positive or negative may vary without departing from the sprit or scope of the described techniques.

In some cases, an instance of training data may include glucose values that are below the hypoglycemic threshold which begin during the day time interval and continue during the night time interval. In such cases, the model manager 902 can be configured to exclude such instances of training data from the training data used to train the machine learning model 408. Alternately, the model manager 902 may include the training data where the hypoglycemic event begins during a day time interval with the training data used to train the machine learning model 408 so that the machine learning model 408 learns this pattern.

As noted above, the machine learning model 408 may be configured to receive additional data 404 as input in addition to an interval of time series glucose measurements. In such implementations, the model manager 902 may form training instances that include the time series of glucose measurements, the respective classification label, and also the additional data 404 describing any other aspects of the user population being used to predict upcoming glucose measurements, e.g., application usage activity, acceleration data, insulin administrations, carbohydrate consumption, exercise, and/or stress. This additional data 404, along with the time series glucose measurements and the classification label, may be processed by the model manger 902 according to one or more known techniques to produce an input vector. This input vector, describing the time series glucose measurements as well as the other aspects, may then be provided to the machine learning model 408. In response, the machine learning model 408 may generate a prediction of upcoming glucose measurements in a similar manner as discussed above, such that the prediction can be compared to the expected classification label of the training instance and weights of the model adjusted based on the comparison.

In one or more implementations, the model manager 902 can detect that a user intervention may have occurred based on the additional data 404. For example, the model manager 902 may detect screen views of a CGM application that indicates the user read an article describing ways to mitigate a hypoglycemic event before the user went to sleep. In this scenario, the user may have taken a subsequent action to mitigate the hypoglycemic event, such as by drinking a glass of juice. This mitigating action, however, may affect the user's glucose levels during the night, such as by preventing a hypoglycemic event. In this case, the instance of training data would be classified as a non-hypoglycemic night because of the intervention taken by the user. The model manager may thus take a variety of approaches. In one or more implementations, the model manager 902 can exclude training data where it is likely, based on the additional data 404, that a user intervention occurred. To do so, the model manager can filter training data based on screen views or some other user action of the additional data. Alternately, instances of training data where the user took a mitigating action may be included with the training data to enable the machine learning model 408 to learn the pattern. Alternately, the model manager may include the additional data 404, such as night time screen views (or other application usage activity) as additional data used to train the machine learning model 408.

As also noted above, management of the machine learning model 408 may include personalizing the machine learning model 408 using transfer learning. In such scenarios, the model manager 902 may initially train the machine learning model 408 at the global level, as described in detail above using instances of training data generated from the data of the user population 110. In transfer learning scenarios, the model manager 902 may then create an instance of this globally trained model for a particular user, such that a copy of the globally trained model is generated for the person 102 and other copies of the globally trained model are generated for other users on a per-user basis.

This globally trained model may then be updated (or further trained) using data specific to the person 102. For example, the model manager 802 may create instances of training data using the glucose measurements 118 of the person 102, and further train the globally trained version of model in a similar manner as described above, e.g., by providing training input portions of the person 102's training data to the machine learning model 408, receiving training predictions of upcoming glucose measurements, comparing those predictions to respective output portions of the training data, and adjusting internal weights of the machine learning model 408. Based on this further training, the machine learning model 408 is trained at a personal level, creating a personally trained machine learning model 408.

It is to be appreciated that the personalizing may be less granular than on a per-user basis, in one or more implementations. For example, the globally trained model may be personalized at a user segment level, i.e., a set of similar users of the user population 110 that is less than an entirety of the user population 110. In this way, the model manager 902 may create copies of the globally trained machine learning model 408 on a per-segment basis and train the global versions at the segment level, creating segment specific machine learning models 408.

In one or more implementations, the model manager 902 may personalize the machine learning model 408 at the server level, e.g., at servers of the CGM platform 112. This model may then be maintained at the server level and/or communicated to the computing device 108, e.g., for integration with an application of the CGM platform 112 at the computing device 108. Alternately or additionally, at least a portion of the model manager 902 may be implemented at the computing device 108, such that the globally trained version of the machine learning model 408 is communicated to the computing device 108 and the transfer learning (i.e., the further training discussed above to personalize the model) is carried out at the computing device 108. Although transfer learning may be leveraged in one or more scenarios, it is to be appreciated that in other scenarios such personalization may not be utilized and the described techniques may be implemented using globally trained versions of the machine learning model 408.

In one or more implementations, the hypoglycemic event prediction system 310 is further configured to identify a recurring pattern of night time hypoglycemia for a user based on the glucose measurements 118 obtained for the user during the night time interval from the CGM system worn by the user. In these instances, the hypoglycemic event prediction system 310 can notify the user of the identified pattern of night time hypoglycemia. In some cases, the user can be notified in cases where the detected night time hypoglycemia is below a particular glucose threshold that corresponds to “severe” hypoglycemia, e.g., less 54 mg/dL. In these scenarios, the user can be asked whether the user would like to receive predictions and alerts for night time hypoglycemia generated by the hypoglycemic event prediction system 310 as described throughout.

As part of this, the hypoglycemic event prediction system 310 may also enable the user to specify the glucose level at which they would like to be notified or alerted. For example, some users may want to receive predictions and alerts when their night time glucose level is predicted to fall below 54 mg/dL, while other users may prefer to receive predictions and alerts when their night time glucose level is predicted to fall below 70 mg/dL. As another example, some users (e.g., users with long term type I diabetes) may wish to receive alerts if their night time glucose level is predicted to fall below a higher threshold, such as 80 mg/dL.

The hypoglycemic event prediction system 310 may be further configured to detect that the user no longer experiences night time hypoglycemia based on the glucose measurements 118 obtained during the night time interval. In this case, the hypoglycemic event prediction system 310 can ask the user if the use would like the predictions and alerts for night time hypoglycemia to be disabled. In this way, the hypoglycemic event prediction system 310 enables better sleep with fewer hypoglycemia alerts which may also increase the likelihood of the user waking-up within range.

Having discussed example details of the techniques for hypoglycemic event prediction using machine learning, consider now some example procedures to illustrate additional aspects of the techniques.

### Example Procedures

This section describes example procedures for hypoglycemic event prediction using machine learning. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a prediction system, such as hypoglycemic event prediction system 310 that makes use of the sequencing manager 406, the machine learning model 408, and the model manager 902.

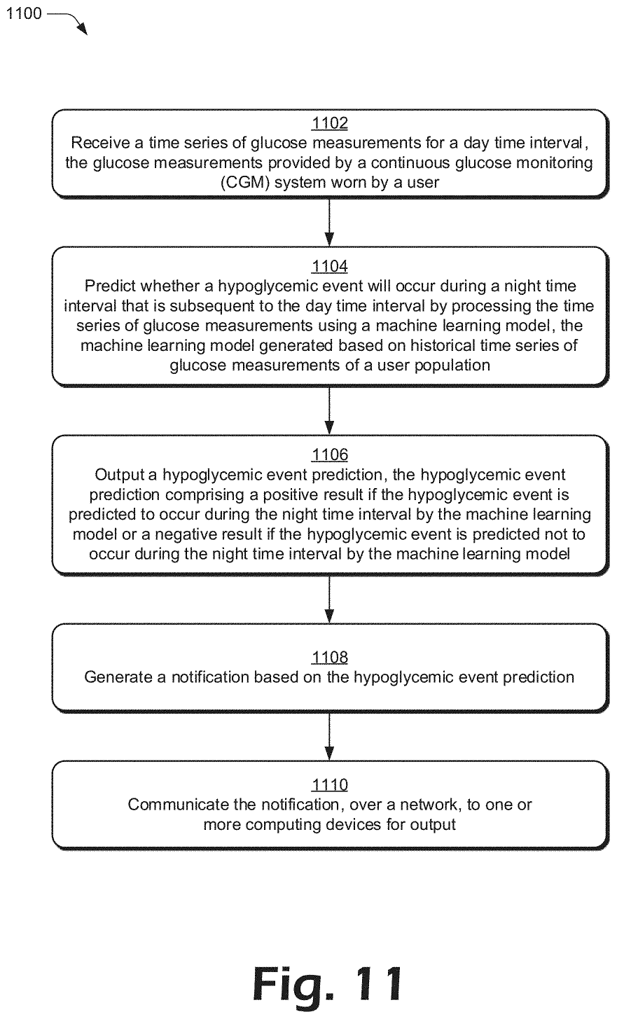

FIG. 11 depicts a procedure 1100 in an example implementation in which a machine learning model predicts whether a hypoglycemic event will occur during a night time interval.

A time series of glucose measurements for a day time interval is received (block 1102). In accordance with the principles discussed herein, the glucose measurements are provided by a continuous glucose monitoring (CGM) system worn by a user. By way of example, the machine learning model 408 receives the time series glucose measurements 412 for a day time interval, and the glucose measurements are provided by the CGM system 104 worn by the person 102. In particular, the CGM system 104 includes the sensor 202, which is inserted subcutaneously into skin of the person 102 and used to measure glucose in the person 102's blood.

The time series of glucose measurements are processed using a machine learning model to predict whether a hypoglycemic event will occur during a night time interval that is subsequent to the day time interval (block 1104). In accordance with the principles discussed herein, the machine learning model is generated based on historical time series of glucose measurements of a user population. By way of example, the machine learning model 408 processes the time series of glucose measurements 412 to generate a hypoglycemic event prediction 312. Generally, the hypoglycemic event prediction 312, output by the machine learning model 408, predicts whether a hypoglycemic event will occur for the user during a night time interval, e.g., that is subsequent to the day time interval of the time series glucose measurements 412. Continuing with the example above, if the time series glucose measurements correspond to a day time interval from 6 AM in the morning to 10 PM at night, then the machine learning model 408 can generate the hypoglycemic event prediction 312 for a night time interval that is subsequent to the day time interview, e.g., from 10 PM that night to 6 AM the following morning. As described throughout, the machine learning model 408 may also obtain additional data 404, and generate the prediction based at least in part on the additional data 404.

A hypoglycemic event prediction is output (block 1106). In accordance with the principles discussed herein, the hypoglycemic event prediction 312 includes a positive result 414 if the hypoglycemic event is predicted to occur during the night time interval by the machine learning model 408 or a negative result 416 if the hypoglycemic event is predicted not to occur during the night time interval by the machine learning model 408. By way of example, the hypoglycemic event prediction system 310 outputs the hypoglycemic event prediction 312, such as for processing by additional logic (e.g., to generate recommendations or notifications), for storing in the storage device 120, for communication to one or more computing devices, or for display, to name just a few.

A notification is generated based on the hypoglycemic event prediction (block 1108). By way of example, the data analytics platform 122 generates the notification 314 based on the hypoglycemic event prediction 312. The notification 314 may include an alert 704 that informs person 102 of the likelihood the person will experience a hypoglycemic event during the upcoming night. For example, the alert may indicate that the user is predicted to experience a hypoglycemic event during the upcoming night if the hypoglycemic event prediction 312 corresponds to the positive result 414. In contrast, the alert may indicate that the user is not predicted to experience the hypoglycemic event during the upcoming night if the hypoglycemic event prediction 312 corresponds to the negative result 416.

The notification 314 may also include one or more recommendations 706. For example, if the machine learning model 408 predicts that the person 102 is likely to experience hypoglycemia during the night, then the notification manager 702 may output one or more recommendations 706 for mitigating the hypoglycemia, such as to drink a glass of juice before sleep, eat a piece of fruit before sleep, set an alarm for a certain time to wake up and drink juice or eat fruit, and so forth. On the other hand, if the machine learning model 408 predicts that the user is not likely to experience hypoglycemia over the upcoming predetermined time period, then the notification manager 702 can output the a notification indicating that this is the case and/or that no mitigating actions need to be taken.

In one or more implementations, the notification 314 may also include a visual representation of the confidence score 418 to inform the user of the accuracy of the prediction. For example, if the machine learning model 408 predicts the occurrence of a hypoglycemic event during the night with 90% confidence, then the notification 314 may visually indicate that this confidence level to the user as part of the alert 704. Alternately, if the machine learning model 408 predicts that the user will not experience an episode of hypoglycemia during the night with 90% confidence, then the notification 314 may visually indicate this confidence level to the user as part of the alert 704.

The notification is communicated, over a network, to one or more computing device for output (block 1110). By way of example, a communication interface of the data analytics platform 122 communicates the notification 314 over the network 116 to the computing device 108 of the person 102, e.g., for output via an application of the CGM platform 112. Alternately or in addition, the data analytics platform 122 communicates the notification 314 over the network 116 to a computing device associated with a health care provider (not shown) and/or a computing device associated with a telemedicine service (not shown), e.g., for output via a provider portal.

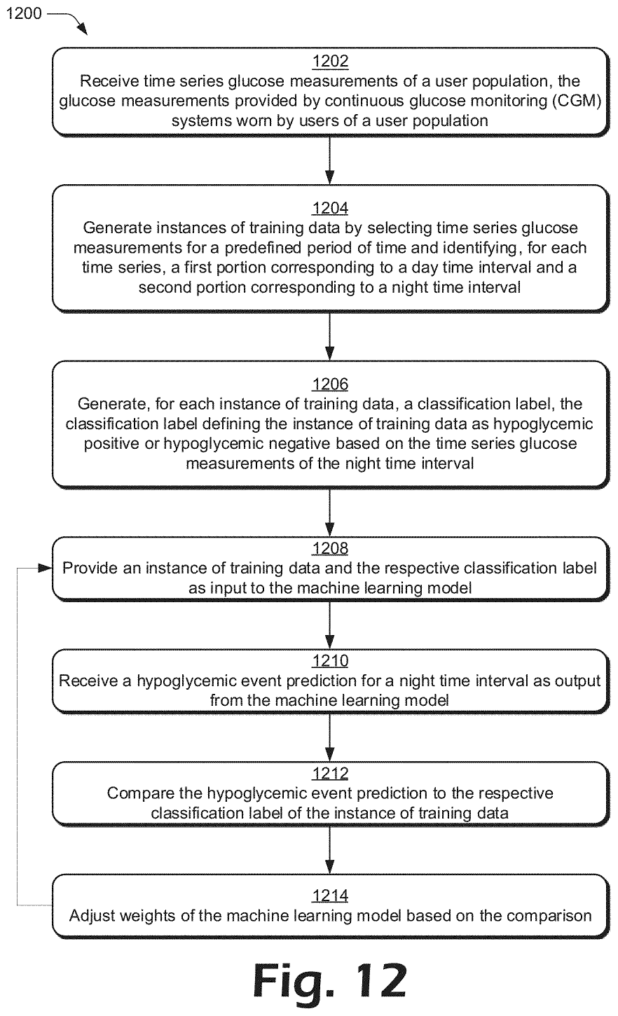

FIG. 12 depicts a procedure 1200 in an example implementation in which a machine learning model is trained to predict hypoglycemic events based on historical time series glucose measurements of a user population.

Time series glucose measurements of a user population are received (block 1202). In accordance with the principles discussed herein, the glucose measurements are provided by CGM systems worn by users of a user population. By way of example, the sequencing manager 406 obtains the glucose measurements 118 of users of the user population 110 and the timestamps 402 of those measurements and forms time series of the user population 110's glucose measurements 118 by ordering the user population 110's glucose measurements 118 according to the respective timestamps 402. The sequencing manager 406 may also interpolate missing measurements, such as measurements missing due to data corruption or communication errors.

Instances of training data are generated by selecting time series glucose measurements for a predefined period of time and identifying, for each time series, a first portion corresponding to a day time interval and a second portion corresponding to a night time interval (block 1204). In accordance with the principles discussed herein, the predefined period of time may correspond to a 24-hour period of time, such that the day time interval corresponds to day time portion of time (e.g., 6 AM to 10 PM) while the night time interval corresponds to a night time portion of time (e.g., 10 PM to 6 AM the following morning). By way of example, the model manager 902 generates instances of training data by selecting time glucose measurements for a 24-hour period of time corresponding to day, and then identifying a first portion of the training time series corresponding to a day time interval and second portion of the training time series corresponding to a night time interval.

A classification label is generated for each instance of training data (block 1206). In accordance with the principles discussed herein, each classification label defines the respective instance of training data as hypoglycemic positive or hypoglycemic negative based on the time series glucose measurements of the night time interval. By way of example, the model manager 902 generates, for each instance of training data, a classification label that defines the instance of training data as hypoglycemic positive or hypoglycemic negative based on the time series glucose measurements of the night time interval. For example, instances of training data with a certain number of glucose values below a defined hypoglycemic threshold (e.g., four consecutive glucose value below 70 mg/dl) are classified as hypoglycemic positive, whereas instances of training data without said number of glucose values below the defined hypoglycemic threshold are classified as hypoglycemic positive. The classification labels, therefore, serve as a ground truth for comparison to the model's output during training.

Here, blocks 1208-1214 may be repeated until a machine learning model is suitably trained, such as until the machine learning model “converges” on a solution, e.g., the internal weights of the model have been suitably adjusted due to training iterations so that the model consistently generates predictions that substantially match the expected output portions. Alternately or in addition, the blocks 1208-1214 may be repeated for a number of instances (e.g., all instances) of the training data.

An instance of training data and the respective classification label is provided as input to the machine learning model (block 1208). By way of example, the model manager 902 provides an instance of training data generated at block 1204 and the respective classification label generated at block 1206 as input to the machine learning model 408.

A hypoglycemic event prediction for a night time interval is received as output from the machine learning model (block 1210). By way of example, the machine learning model 408 generates a hypoglycemic event prediction for the night time interval, such as by predicting that a hypoglycemic event will occur or not occur during the night time interval.

The hypoglycemic event prediction is compared to the respective classification label of the instance of training data (block 1212). By way of example, the model manager compares the hypoglycemic event prediction generated at block 1210 to the respective classification label of the training instance generated at block 1206, e.g., by using a loss function such as mean squared error (MSE). It is to be appreciated that the model manager 902 may use other loss functions during training, to compare the predictions of the machine learning model 408 to the expected output, without departing from the spirit or scope of the described techniques.

Weights of the machine learning model are adjusted based on the comparison (block 1214). By way of example, the model manager 902 may adjust internal weights of the machine learning model 408 based on the comparing so that the machine learning model can substantially reproduce the expected classification label (e.g., whether night time hypoglycemia will occur) when glucose traces for a day time interval are provided as input in the future.

Having described example procedures in accordance with one or more implementations, consider now an example system and device that can be utilized to implement the various techniques described herein.

### Example System and Device

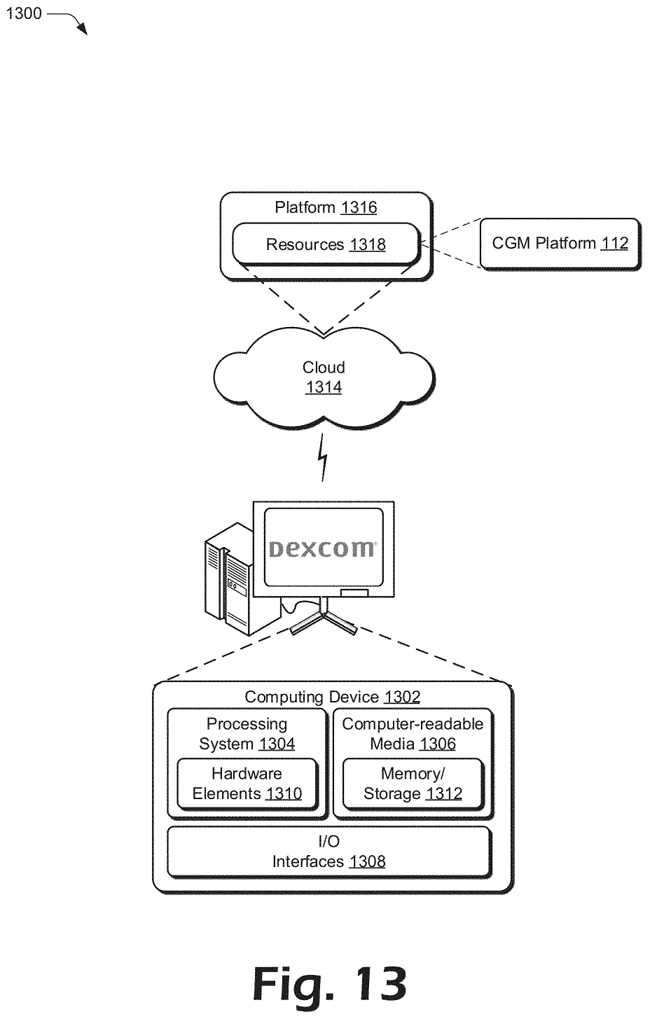

FIG. 13 illustrates an example system generally at 1300 that includes an example computing device 1302 that is representative of one or more computing systems and/or devices that may implement the various techniques described herein. This is illustrated through inclusion of the CGM platform 112. The computing device 1302 may be, for example, a server of a service provider, a device associated with a client (e.g., a client device), an on-chip system, and/or any other suitable computing device or computing system.

The example computing device 1302 as illustrated includes a processing system 1304, one or more computer-readable media 1306, and one or more I/O interfaces 1308 that are communicatively coupled, one to another. Although not shown, the computing device 1302 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1304 is representative of functionality to perform one or more operations using hardware. Accordingly, the processing system 1304 is illustrated as including hardware elements 1310 that may be configured as processors, functional blocks, and so forth. This may include implementation in hardware as an application specific integrated circuit or other logic device formed using one or more semiconductors. The hardware elements 1310 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1306 is illustrated as including memory/storage 1312. The memory/storage 1312 represents memory/storage capacity associated with one or more computer-readable media. The memory/storage component 1312 may include volatile media (such as random access memory (RAM)) and/or nonvolatile media (such as read only memory (ROM), Flash memory, optical disks, magnetic disks, and so forth). The memory/storage component 1312 may include fixed media (e.g., RAM, ROM, a fixed hard drive, and so on) as well as removable media (e.g., Flash memory, a removable hard drive, an optical disc, and so forth). The computer-readable media 1306 may be configured in a variety of other ways as further described below.

Input/output interface(s) 1308 are representative of functionality to allow a user to enter commands and information to computing device 1302, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1302 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1302. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1302, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1310 and computer-readable media 1306 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1310. The computing device 1302 may be configured to implement particular instructions and/or functions corresponding to the software and/or hardware modules. Accordingly, implementation of a module that is executable by the computing device 1302 as software may be achieved at least partially in hardware, e.g., through use of computer-readable storage media and/or hardware elements 1310 of the processing system 1304. The instructions and/or functions may be executable/operable by one or more articles of manufacture (for example, one or more computing devices 1302 and/or processing systems 1304) to implement techniques, modules, and examples described herein.

The techniques described herein may be supported by various configurations of the computing device 1302 and are not limited to the specific examples of the techniques described herein. This functionality may also be implemented all or in part through use of a distributed system, such as over a “cloud” 1314 via a platform 1316 as described below.

The cloud 1314 includes and/or is representative of a platform 1316 for resources 1318. The platform 1316 abstracts underlying functionality of hardware (e.g., servers) and software resources of the cloud 1314. The resources 1318 may include applications and/or data that can be utilized while computer processing is executed on servers that are remote from the computing device 1302. Resources 1318 can also include services provided over the Internet and/or through a subscriber network, such as a cellular or Wi-Fi network.

The platform 1316 may abstract resources and functions to connect the computing device 1302 with other computing devices. The platform 1316 may also serve to abstract scaling of resources to provide a corresponding level of scale to encountered demand for the resources 1318 that are implemented via the platform 1316. Accordingly, in an interconnected device embodiment, implementation of functionality described herein may be distributed throughout the system 1300. For example, the functionality may be implemented in part on the computing device 1302 as well as via the platform 1316 that abstracts the functionality of the cloud 1314.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
generating instances of training data by selecting time series glucose measurements of a user population for a predefined period of time, identifying, for each time series, a first portion corresponding to a day time interval and a second portion corresponding to a night time interval;
generating, for each instance of training data, a classification label, the classification label defining the instance of training data as hypoglycemic positive or hypoglycemic negative based on the time series glucose measurements of the night time interval; and
training a machine learning model to predict a hypoglycemic event during the night time interval using the generated instances of training data and the classification labels.

2. The method of claim 1, wherein the training the machine learning model to predict the hypoglycemic event during the night time interval further comprises:
providing the instances of training data and the respective classification labels to the machine learning model;
receiving, for each instance of training data, a hypoglycemic event prediction for a night time interval from the machine learning model;
comparing the hypoglycemic event prediction to the classification label of the instance of training data; and
adjusting weights of the machine learning model based on the comparison.

3. The method of claim 1, further comprising classifying each instance of training data as hypoglycemic positive if there are a predefined number of glucose values during the night time interval which are below a hypoglycemic threshold.

4. The method of claim 3, further comprising classifying each instance of training data as hypoglycemic negative if there are not the predefined number of glucose values during the night time interval which are below the hypoglycemic threshold.

5. The method of claim 1, further comprising predicting a hypoglycemic event using the trained machine learning model by:
receiving, as input, time series glucose measurements for a day time interval;
generating predicted time series glucose measurements for a corresponding night time interval based on the input; and
generating the hypoglycemic event prediction based on the predicted time series measurements for the night time interval.

6. The method of claim 1, wherein the predefined period of time corresponds to a 24-hour period of time.

7. The method of claim 1, wherein the machine learning model comprises a neural network.

8. The method of claim 1, wherein the glucose measurements are obtained from wearable glucose monitoring systems worn by users of the user population.

9. The method of claim 8, wherein the wearable glucose monitoring system include a plurality of continuous glucose monitoring (CGM) systems worn by users of the user population.

10. A system comprising:
a storage device to maintain glucose measurements and additional data associated with a user; and
a machine learning model to predict whether a hypoglycemic event will occur during a night time interval that is subsequent a day time interval, the prediction generated responsive to receipt of a time series of the glucose measurements corresponding to the day time interval and additional data by the neural network as input, and the neural network trained based on historical time series of glucose measurements and historical additional data of a user population.

11. The system as described in claim 10, wherein the machine learning model comprises a neural network.

12. The system as described in claim 11, further comprising a model manager configured to train the neural network using the historical time series of glucose measurements.

13. The system as described in claim 12, wherein the model manager is further configured to train the neural network using the historical additional data of the user population.

14. The system as described in claim 10, wherein the storage device is further configured to maintain the historical time series of glucose measurements and the historical additional data of the user population.

15. The system as described in claim 10, wherein the glucose measurements are obtained from a wearable glucose monitoring device worn by the user.

16. The system as described in claim 15, wherein the wearable glucose monitoring device comprises a continuous glucose monitoring (CGM) system worn by the user.

17. One or more computer-readable storage media having instructions stored thereon that are executable by one or more processors to perform operations comprising:
generating instances of training data by selecting time series glucose measurements of a user population for a predefined period of time, identifying, for each time series, a first portion corresponding to a day time interval and a second portion corresponding to a night time interval;
generating, for each instance of training data, a classification label, the classification label defining the instance of training data as hypoglycemic positive or hypoglycemic negative based on the time series glucose measurements of the night time interval; and
training a machine learning model to predict a hypoglycemic event during the night time interval using the generated instances of training data and the classification labels.

18. The one or more computer-readable storage media of claim 17, wherein the training the machine learning model to predict the hypoglycemic event further comprises:
providing the instances of training data and the respective classification labels to the machine learning model;
receiving, for each instance of training data, a hypoglycemic event prediction for a night time interval from the machine learning model;
comparing the hypoglycemic event prediction to the classification label of the instance of training data; and
adjusting weights of the machine learning model based on the comparison.

19. The one or more computer-readable storage media of claim 17, wherein the operations further comprise classifying each instance of training data as hypoglycemic positive if there are a predefined number of glucose values during the night time interval which are below a hypoglycemic threshold.

20. The one or more computer-readable storage media of claim 19, wherein the operations further comprise classifying each instance of training data as hypoglycemic negative if there are not the predefined number of glucose values during the night time interval which are below the hypoglycemic threshold.

21. The one or more computer-readable storage media of claim 17, wherein the operations further comprise predicting a hypoglycemic event using the trained machine learning model by:
receiving, as input, time series glucose measurements for a day time interval;
generating predicted time series glucose measurements for a corresponding night time interval based on the input; and
generating the hypoglycemic event prediction based on the predicted time series measurements for the night time interval.

22. The one or more computer-readable storage media of claim 17, wherein the predefined period of time corresponds to a 24-hour period of time.

23. The one or more computer-readable storage media of claim 17, wherein the machine learning model comprises a neural network.

24. The one or more computer-readable storage media of claim 17, wherein the glucose measurements are obtained from wearable glucose monitoring systems worn by users of the user population.

25. The one or more computer-readable storage media of claim 24, wherein the wearable glucose monitoring systems include a plurality of continuous glucose monitoring (CGM) systems worn by users of the user population.
